(19) United States
(12) Patent Application Publication
(10) Pub. No.: US 2021/0230532 A1
(43) Pub. Date: Jul. 29, 2021

(54) SYSTEMS AND METHODS FOR SCALABLE MANUFACTURING OF THERAPEUTIC CELLS IN BIOREACTORS

(71) Applicant: PBS Biotech, Inc., Camarillo, CA (US)

(72) Inventors: Chanyong Brian Lee, Thousand Oaks, CA (US); Breanna Shalyn Borys, Thousand Oaks, CA (US)

(21) Appl. No.: 17/160,139

(22) Filed: Jan. 27, 2021

Related U.S. Application Data
(60) Provisional application No. 62/966,441, filed on Jan. 27, 2020.

(51) Int. Cl. C12M 1/34; C12N 5/0775; C12N 5/0735; C12N 5/074; C12M 1/06; C12M 1/42

(52) U.S. Cl. CPC C12M 41/42; C12N 5/0662; C12N 5/0606; C12N 5/0696; C12N 2531/00; C12M 35/04; C12M 27/06; C12N 2511/00; C12N 2527/00; C12M 27/02

(57) ABSTRACT

Systems and methods for scalable manufacturing of therapeutic cells in bioreactors are disclosed. Fluid dynamic considerations for scale in accordance with an implementation include a method of production of therapeutic cells grown on microcarriers or as cell aggregates in a suspension-based bioreactor includes depositing a suspension comprising cells suspended in a volume of culture fluid into a bioreactor and setting an agitation rate of a mixer disposed in the bioreactor. The method includes actuating the mixer at the set agitation rate to mix the suspension in the bioreactor. The suspension includes a plurality of turbulent eddies generated by the mixer. A magnitude of an energy dissipation rate (EDR) of at least approximately 60% of the turbulent eddies can be less than approximately 0.0015 m2/s3.

### CROSS-REFERENCE TO RELATED APPLICATION

Priority is claimed to U.S. Provisional Patent Application No. 62/966,441, filed Jan. 27, 2020, the entire contents of which are incorporated by reference herein.

### FIELD OF THE DISCLOSURE

This disclosure relates generally to the production of therapeutic cells in bioreactors and, more specifically, to systems and methods for scalable manufacturing of therapeutic cells in bioreactors.

### BACKGROUND

With the potential to cure numerous types of serious disease indications, cell therapies are poised to revolutionize the biopharmaceutical industry. An increasing number of allogeneic therapeutic cell candidates are currently in development or entering early stages of clinical trials. However, large-scale manufacturing of these therapeutic cell products, sufficient to meet future commercial demand, has yet to be developed and demonstrated.

The limitation of using 2D manufacturing platforms for commercial production of therapeutic cells is well recognized by the biopharmaceutical industry. The primary cost of goods for 2D manufacturing, namely expensive capital investments and labor costs, would become prohibitive at commercial scale. Instead, single-use bioreactors as a 3D manufacturing platform are widely considered to be the technology used for scalable therapeutic cell manufacturing.

### SUMMARY

In accordance with a first implementation, a method of scaling production of therapeutic cells grown on microcarriers or as cell aggregates in a suspension-based bioreactor includes determining a target average energy dissipation rate (EDR) of turbulent eddies within a suspension including cells disposed in a small scale bioreactor. The method includes determining a small scale agitation rate to achieve the target average EDR in the small scale bioreactor and determining a large scale agitation rate to achieve the target average EDR in a large scale bioreactor. The large scale agitation rate is directly dependent on the small scale agitation rate. The method includes depositing a suspension comprising a plurality of cells suspended in a volume of culture fluid into the large scale bioreactor and setting an agitation rate of a mixer disposed in the large scale bioreactor to the large scale agitation rate. The method includes actuating the mixer in the large scale bioreactor at the large scale agitation rate to mix the suspension with an average EDR approximately equal to the target average EDR.

In accordance with a second implementation, a method of operating a large scale suspension-based bioreactor for the production of cells grown on microcarriers or as cell aggregates includes selecting a large scale bioreactor for production of cells grown on microcarriers or as cell aggregates. The large scale bioreactor has a large scale mixer in a large scale vessel. The method includes determining a large scale agitation rate for the large scale mixer. The large scale agitation rate is determined based on a small scale agitation rate of a small scale mixer in a small scale vessel of a small scale bioreactor that achieves a target average energy dissipation rate (EDR) of turbulent eddies in a suspension in the small scale bioreactor. The method includes depositing a suspension comprising cells suspended in a volume of culture fluid into the large scale bioreactor and setting the agitation rate of the large scale mixer to the large scale agitation rate. The method includes actuating the large scale mixer at the large scale agitation rate to mix the cells in the suspension at an average EDR approximately equal to the target average EDR.

In accordance with a third implementation, a large scale suspension-based system for the production of cells grown on microcarriers or as cell aggregates includes a bioreactor and a suspension. The bioreactor includes a vessel and a mixer disposed in the vessel. The mixer is operably coupled to a drive mechanism and is operated at an agitation rate. The suspension includes cells suspended in a volume of culture fluid disposed in the vessel and being mixed by the mixer. The suspension includes a plurality of turbulent eddies generated by the mixer. The plurality of turbulent eddies each have an energy dissipation rate (EDR). A magnitude of the EDR of at least approximately 60%, at least approximately 70%, at least approximately 75%, at least approximately 80%, at least approximately 85%, at least approximately 90%, at least approximately 95%, or at least approximately 97% of the turbulent eddies is less than approximately 0.0015 m²/s³.

In accordance with a fourth implementation, a method of production of therapeutic cells grown on microcarriers or as cell aggregates in a suspension-based bioreactor includes depositing a suspension comprising cells suspended in a volume of culture fluid into a bioreactor and setting an agitation rate of a mixer disposed in the bioreactor. The method includes actuating the mixer at the set agitation rate to mix the suspension in the bioreactor. The suspension includes a plurality of turbulent eddies generated by the mixer. A magnitude of an energy dissipation rate (EDR) of at least approximately 60%, at least approximately 70%, at least approximately 75%, at least approximately 80%, at least approximately 85%, at least approximately 90%, at least approximately 95%, or at least approximately 97% of the turbulent eddies is less than approximately 0.0015 m²/s³.

In accordance with a fifth implementation, a bioreactor system includes a containment vessel, a mixer, and a processor. The containment vessel defines a first working volume and the mixer is in the containment vessel and configured to rotate about an axis so as to stir contents of the containment vessel. The processor is adapted to access a first agitation rate at which a mixer of a second bioreactor having a second working volume is operated. Operating the mixer of the second bioreactor at the first agitation rate achieves an average energy dissipation rate (average EDR) of turbulent eddies within a suspension including cells disposed in the second bioreactor. Based on the first agitation rate, the processor is adapted to determine a second agitation rate at which the mixer in the containment vessel is configured to operate to substantially achieve a target average EDR of turbulent eddies within a suspension including cells in the containment vessel. The target average EDR is approximately equal to the average EDR. The processor is adapted to cause the mixer of the containment vessel to rotate at the second agitation rate.

In accordance with a sixth implementation, a bioreactor for growing therapeutic pluripotent stem cells derived from humans or animals on microcarriers and/or in aggregates. The microcarriers and/or aggregates are suspended in the culture fluid using an average power input per mass level of 3.5 cm²/sec³ or less.

In further accordance with the foregoing first, second, third, fourth, fifth, and/or sixth implementations, an apparatus and/or method may further include or comprise any one or more of the following:

In accordance with an implementation, the average EDR includes an average of a plurality of actual EDR data points within the volume of the suspension in the large scale bioreactor. A magnitude of at least approximately 60%, at least approximately 70%, at least approximately 75%, at least approximately 80%, at least approximately 85%, at least approximately 90%, at least approximately 95%, or at least approximately 97% of the plurality of actual EDR data points is less than approximately 0.0015 m²/s³.

In accordance with another implementation, at least one of the small scale and large scale agitation rates is in a range between approximately 0 rpm and approximately 120 rpm.

In accordance with another implementation, at least one of the small scale and large scale agitation rates are in a range between approximately 12 rpm and approximately 77 rpm.

In accordance with another implementation, the target average EDR is in a range between approximately 0 m²/s³ and approximately 0.006 m²/s³.

In accordance with another implementation, the target average EDR is in a range between approximately 0.0003 m²/s³ and approximately 0.0015 m²/s³.

In accordance with another implementation, actuating the mixer in the large scale bioreactor comprises actuating a vertical wheel mixer having a horizontal axis of rotation.

In accordance with another implementation, actuating the mixer in the large scale bioreactor comprises actuating a mixer having a vertical axis of rotation.

In accordance with another implementation, depositing a suspension including cells into the large scale bioreactor comprises depositing pluripotent stem cells (PSCs) into the large scale bioreactor.

In accordance with another implementation, the method further includes depositing microcarriers into the large scale bioreactor.

In accordance with another implementation, the large scale bioreactor has a volume larger than a volume of the small scale bioreactor.

In accordance with another implementation, the average EDR includes an average of a plurality of actual EDR data points within the volume of the suspension in the large scale bioreactor. A magnitude of at least approximately 60%, at least approximately 70%, at least approximately 75%, at least approximately 80%, at least approximately 85%, at least approximately 90%, at least approximately 95%, or at least approximately 97% of the plurality of actual EDR data points is less than approximately 0.0015 m²/s³.

In accordance with another implementation, at least one of the small scale and large scale agitation rates is in a range between approximately 0 rpm and approximately 120 rpm.

In accordance with another implementation, at least one of the small scale and large scale agitation rates is in a range between approximately 12 rpm and approximately 77 rpm.

In accordance with another implementation, the target average EDR is in a range between approximately 0 m²/s³ and approximately 0.006 m²/s³.

In accordance with another implementation, the target average EDR is in a range between approximately 0.0003 m²/s³ and approximately 0.0015 m²/s³.

In accordance with another implementation, actuating the large scale mixer comprises a vertical wheel mixer having a horizontal axis of rotation.

In accordance with another implementation, actuating the large scale mixer comprises actuating a mixer having a vertical axis of rotation.

In accordance with another implementation, depositing a suspension including cells into the large scale bioreactor comprises depositing pluripotent stem cells (PSCs) into the large scale bioreactor.

In accordance with another implementation, the method further includes depositing microcarriers into the large scale bioreactor.

In accordance with another implementation, selecting a large scale bioreactor comprises selecting a bioreactor with a volume larger than a volume of the small scale bioreactor.

In accordance with another implementation, the target average EDR is in a range between approximately 0 m²/s³ and approximately 0.006 m²/s³.

In accordance with another implementation, the target average EDR is in a range between approximately 0.0003 m²/s³ and approximately 0.0015 m²/s³.

In accordance with another implementation, the vessel has a volume at least one of between approximately 0.1 L and approximately 500 L or between approximately 0.1 L and approximately 2000 L.

In accordance with another implementation, the mixer includes a vertical wheel mixer having a horizontal axis of rotation.

In accordance with another implementation, the vessel includes a curved bottom wall.

In accordance with another implementation, the mixer includes a vertical axis of rotation.

In accordance with another implementation, the cells include pluripotent stem cells (PSCs).

In accordance with another implementation, the system further includes microcarriers in the suspension.

In accordance with another implementation, the target average EDR is in a range between approximately 0 m²/s³ and approximately 0.006 m²/s³.

In accordance with another implementation, the target average EDR is in a range between approximately 0.0003 m²/s³ and approximately 0.0015 m²/s³.

In accordance with another implementation, the method further includes selecting the bioreactor from a plurality of available bioreactors, each comprising a volume at least one of between approximately 0.1 L and approximately 500 L or between approximately 0.1 L and approximately 2000 L.

In accordance with another implementation, the mixer includes a vertical wheel mixer having a horizontal axis of rotation.

In accordance with another implementation, the vessel includes a curved bottom wall.

In accordance with another implementation, the mixer includes a vertical axis of rotation.

In accordance with another implementation, depositing a suspension includes cells suspended in a volume of a culture fluid into the bioreactor comprises depositing pluripotent stem cells (PSCs) into the bioreactor.

In accordance with another implementation, the method includes depositing microcarriers into the bioreactor.

In accordance with another implementation, the bioreactor system further includes a user interface adapted to receive an input associated with the first agitation rate. The user interface being operatively coupled to the processor.

In accordance with another implementation, the first working volume is greater than the second working volume.

In accordance with another implementation, the containment vessel has a working volume at least one of between approximately 0.1 L and approximately 500 L or between approximately 0.1 L and approximately 2000 L.

In accordance with another implementation, the containment vessel has walls including a lower curved wall located at a lower end of the vessel.

In accordance with another implementation, the mixer is configured to rotate about a horizontal axis.

In accordance with another implementation, the mixer is configured to rotate about a vertical axis.

In accordance with another implementation, the target average EDR is in a range between approximately 0 m²/s³ and approximately 0.006 m²/s³.

In accordance with another implementation, the target average EDR is in a range between approximately 0.0003 m²/s³ and approximately 0.0015 m²/s³.

In accordance with another implementation, the bioreactor system further includes the suspension including cells disposed in the containment vessel.

In accordance with another implementation, the cells include pluripotent stem cells (PSCs).

In accordance with another implementation, the bioreactor system further includes microcarriers in the suspension.

In another implementation, the microcarriers or aggregates have an average diameter of 100 microns or greater.

In another implementation, over 90% of the bioreactor volume is below a target average energy dissipation rate (EDR), where EDR is the energy dissipation rate per unit mass, typically measured or predicted in units of m²/s³ or cm²/s³, for the culture fluid undergoing fluid flow.

In another implementation, over 99% of the bioreactor volume is below a target average EDR.

In another implementation, over 90% of the bioreactor volume is below a target average EDR of 1.30E-2 m²/s³.

In another implementation, over 99% of the bioreactor volume is below a target average EDR of 1.30E-2 m²/s³.

In another implementation, this property is maintained upon scale up in series of increasingly larger bioreactors.

In another implementation, the increase in scale in bioreactor working volumes goes from 100 mls up to 3 liters, 100 mls up to 15 liters, 100 mls up to 80 liters, 100 mls up to 500 liters, or up to 2000 liters.

In another implementation, the microcarriers or aggregates have an average diameter of 100 microns or greater.

In another implementation, a bioreactor with properties from the implementations disclosed above and/or below which together result in formation of uniformly spherical cell aggregates of same or similar diameter.

In another implementation, a method of precise control of spherical cell aggregate diameter by changing agitation speed of mixing mechanism.

In another implementation, the properties are maintained during scale up into larger volumes as described in the implementations above and/or below.

In another implementation, the method includes uniformity of cell aggregates size/diameter improves expansion efficiency of cell aggregates.

In another implementation, optimal aggregate diameter can vary by cell type.

### BRIEF DESCRIPTION OF THE DRAWINGS

FIG. 1 illustrates images of MSCs attached to the surface of suspended microcarriers using different fluorescent staining methods.

FIG. 2 shows the growth of PSCs as suspended cell aggregates in a vertical-wheel bioreactor.

FIG. 3 show imaging results directed toward the differentiation of human iPSCs to cerebellar organoids in a vertical-wheel bioreactor having about a 0.1 liter (L) scale.

FIG. 4 illustrates a schematic diagram of an implementation of a system in accordance with the teachings of this disclosure.

FIG. 5 is an isometric view of the mixer that can be used with the bioreactors of FIG. 4.

FIG. 6 illustrates a flowchart for a method of scaling production of therapeutic cells grown on microcarriers or as cell aggregates in a suspension-based bioreactor using the system, the first bioreactor, and/or the second bioreactor of FIG. 4 or any of the other implementations disclosed herein.

FIG. 7 illustrates another flowchart for a method of scaling production of therapeutic cells grown on microcarriers or as cell aggregates in a suspension-based bioreactor using the system, the first bioreactor, and/or the second bioreactor of FIG. 4 or any of the other implementations disclosed herein.

FIG. 8 illustrates another flowchart for a method of scaling production of therapeutic cells grown on microcarriers or as cell aggregates in a suspension-based bioreactor using the system, the first bioreactor, and/or the second bioreactor of FIG. 4 or any of the other implementations disclosed herein.

FIG. 9A illustrates a schematic representation of eddy streams and microcarriers with attached viable cells when the eddy streams are larger than the microcarriers.

FIG. 9B illustrates a schematic representation of one of the microcarriers with attached viable cells and eddy streams that are smaller than the microcarriers.

FIG. 10 is a schematic illustration of the second bioreactor of FIG. 4 and a jig coupled to the second bioreactor and configured to measure impeller power inputs.

FIG. 11 is a graph including an X-axis representing agitation of the wheel of the second bioreactor and a Y-axis representing power per mass.

FIG. 12 is a graph including an X-axis representing Reynolds numbers and a Y-axis representing impeller power.

FIG. 13 is a graph including an X-axis representing Kolmogorov Length Scale (μm) and a Y-axis representing the relative net growth rate.

FIG. 14 is a graph resenting sets of results from microcarrier suspension studies where the x-axis represents revolutions per minute (RPMs).

FIG. 15 is a graph including an X-axis mentioning different bioreactors and the associated agitation rates (rpms) and a Y-axis representing the average power per mass levels required for microcarriers suspension in various bioreactors

FIG. 16 shows results of a computational fluid dynamics (CFD) analysis performed on the first bioreactor having a volume of approximately 3 L.

FIG. 17 shows additional results obtained from computational fluid dynamics (CFD) analysis using the first bioreactor and/or the second bioreactor of FIG. 4.

FIG. 18 is a graph including an X-axis representing the size of cell aggregates and a Y-axis representing the number of cell aggregates.

FIG. 19 is a graph including an X-axis representing the size of PSC aggregates and a Y-axis representing the number of cell aggregates.

FIG. 20A shows a top view of computational fluid dynamics (CFD) analysis results for a Lemniscate liquid flow pattern and velocity stream lines for the second bioreactor.

FIG. 20B shows a side isometric view of computational fluid dynamics (CFD) analysis results for a Lemniscate liquid flow pattern and velocity stream lines for the second bioreactor.

FIG. 21A is a graph including an X-axis representing flow time in seconds and a Y-axis representing velocity.

FIG. 21B is a graph including an X-axis representing flow time in seconds and a Y-axis representing sheer stress.

FIG. 21C is a graph including an X-axis representing flow time in seconds and a Y-axis representing EDR.

FIG. 22A shows computational fluid dynamics (CFD) analysis results related to velocity.

FIG. 22B shows computational fluid dynamics (CFD) analysis results related to sheer stress.

FIG. 22C shows computational fluid dynamics (CFD) analysis results related to energy dissipation.

FIG. 23 illustrates results obtained when growing cells in a bioreactor including a vertical wheel and a bioreactor including a horizontal blade at different agitations rates.

FIG. 24 is a graph including an X-axis representing energy dissipation rates (EDRs) and a Y-axis representing volume percent.

FIG. 25A shows graphs of scale-up trendline equations when using the second bioreactor having a volume of approximately 0.1 L.

FIG. 25B shows graphs including a first line associated with results obtained using the second bioreactor, a second line associated with results obtained using a NDS bioreactor having a horizontal-blade spinner, and a third line associated with results obtained using a DasGip® bioreactor having a horizontal-blade spinner.

FIG. 26A is a graph including an X-axis representing energy dissipation rates (EDRs) and a Y-axis representing a volume average energy dissipation rate.

FIG. 26B is a graph including an X-axis representing the volume of the containment vessel and a Y-axis representing the agitation rate (RPM).

FIG. 26C is a graph including an X-axis representing the agitation rate and a Y-axis representing the average sheer stress.

FIG. 26D is a graph including an X-axis representing the agitation rate and a Y-axis representing the volume average velocity.

FIG. 26E is a graph including an X-axis representing the agitation rate and a Y-axis representing the volume percent.

FIG. 26F shows a more detailed view of a portion of the graph of FIG. 26E.

FIG. 27 is a graph including an X-axis representing the agitation rate and a Y-axis representing the volume percent.

FIG. 28 shows the biological results obtained through the combination of having target volume average EDR inside a threshold range, as well as having majority or at least some of EDR values below the upper threshold value of approximately 1.5E-03 m²/s³.

FIG. 29A is a graph including an X-axis representing time in days and a Y-axis representing viable cells in mL.

FIG. 29B is a graph representing results from the experiments performed in association with FIG. 29A and includes an X-axis representing the agitation rates at which the wheel of the second bioreactor was operated and a y-axis representing the average day 7 aggregate diameter.

FIG. 29C are image results representing results from the experiments performed in association with FIGS. 29A and 29B.

### DETAILED DESCRIPTION

Although the following text discloses a detailed description of implementations of methods, apparatuses and/or articles of manufacture, it should be understood that the legal scope of the property right is defined by the words of the claims set forth at the end of this patent. Accordingly, the following detailed description is to be construed as examples only and does not describe every possible implementation, as describing every possible implementation would be impractical, if not impossible. Numerous alternative implementations could be implemented, using either current technology or technology developed after the filing date of this patent. It is envisioned that such alternative implementations would still fall within the scope of the claims.

Example systems and methods for controlling fluid dynamic conditions in bioreactors are disclosed in order to optimize suspension cell culture processes involving cells grown on microcarriers or as aggregates. These example systems and methods are applicable across a broad range of bioreactor sizes, from approximately 0.1 L working volume for small-scale R&D use to approximately 500 L working volume for large-scale clinical or commercial manufacturing. However, any size bioreactor may be used in accordance with the teachings of this disclosure. For example, the large-scale bioreactor made and/or operated in accordance with the teachings of this disclosure may have a working volume of approximately 2000 L. However, the teachings of this disclosure may be used in association with any size bioreactor, including, for example, a bioreactor having a working volume of approximately 200 L, a bioreactor having a working volume of approximately 300 L, a bioreactor having a working volume of approximately 400 L, a bioreactor having a working volume of approximately 600 L, a bioreactor having a working volume of approximately 700 L, a bioreactor having a working volume of approximately 800 L, a bioreactor having a working volume of approximately 900 L, a bioreactor having a working volume of approximately 1000 L, a bioreactor having a working volume of approximately 1100 L, a bioreactor having a working volume of approximately 1200 L, a bioreactor having a working volume of approximately 1300 L, a bioreactor having a working volume of approximately 1400 L, a bioreactor having a working volume of approximately 1500 L, a bioreactor having a working volume of approximately 1600 L, a bioreactor having a working volume of approximately 1700 L, a bioreactor having a working volume of approximately 1800 L, a bioreactor having a working volume of approximately 1900 L, a bioreactor having a working volume of approximately 2100 L, etc.

In order for bioreactors to become a standard manufacturing platform for therapeutic cells, suspension-based cell culture processes developed in a small-scale bioreactor are to be demonstrated in a repeatable way at larger scales in accordance with the teachings of this disclosure. Providing a threshold growth environment for cells inside bioreactors may be done to increase cell yield while maintaining threshold quality attributes, and to demonstrate the feasibility of commercial-scale production for therapeutic cell products.

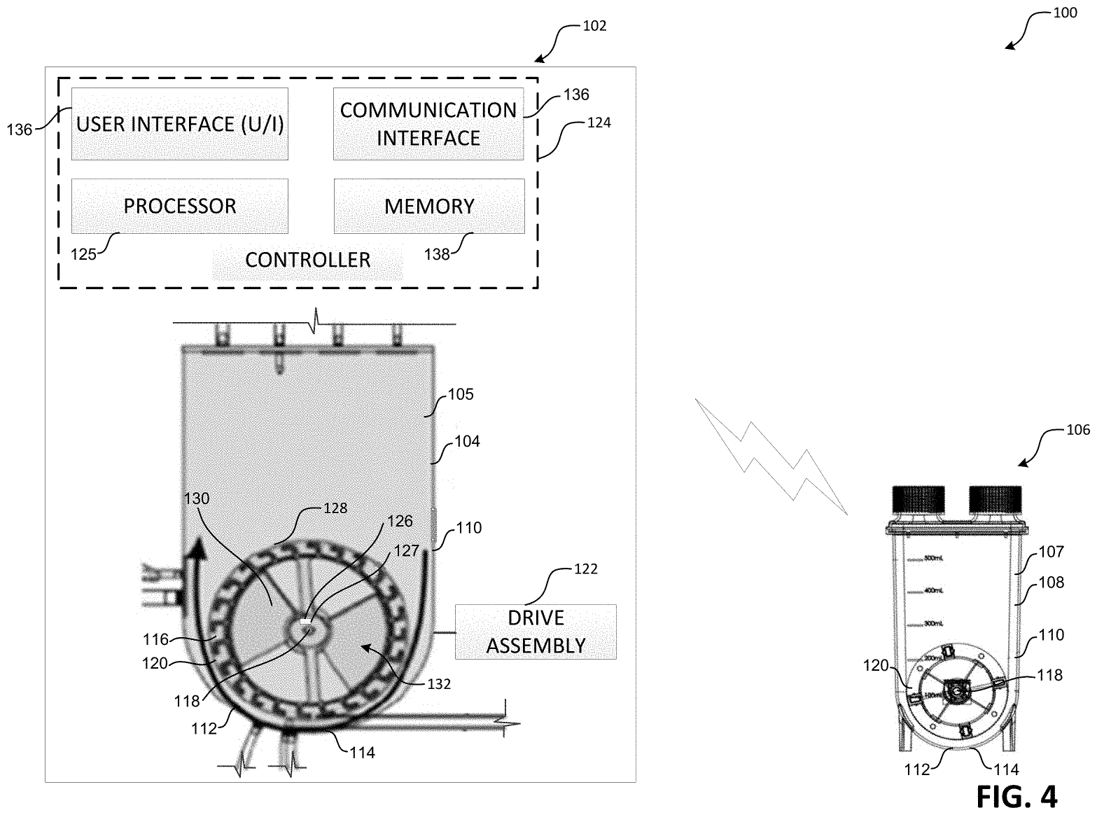

Most allogeneic therapeutic cells are anchorage-dependent and therefore are attached to a surface to proliferate. Different anchorage-dependent cell types vary significantly in their requirements and behavior within bioreactor-based suspension cultures. Some examples of these cell types include human primary cells and mesenchymal stem cells (MSCs) that are typically grown on the surface of plastic microcarriers that are suspended inside the bioreactor (FIG. 4). Related cell products include extracellular vesicles, such as exosomes, that can be produced from MSCs on microcarriers.

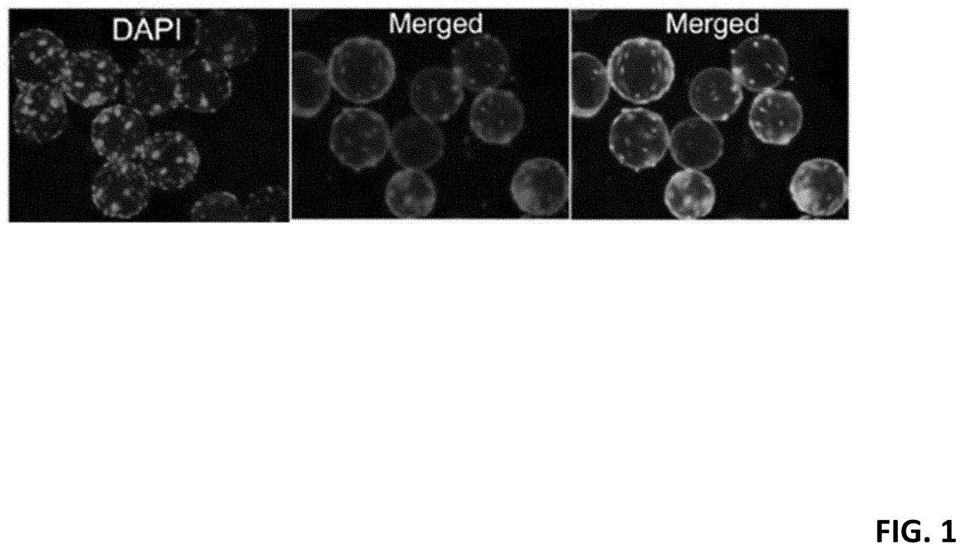

FIG. 1 illustrates images of MSCs attached to the surface of suspended microcarriers using different fluorescent staining methods.

Pluripotent stem cells (PSCs), which encompass types such as embryonic stem cells (ESCs) or induced pluripotent stem cells (iPSCs), naturally clump together to form spherical cell aggregates during both cell expansion and directed differentiation processes and, thus, do not require microcarriers.

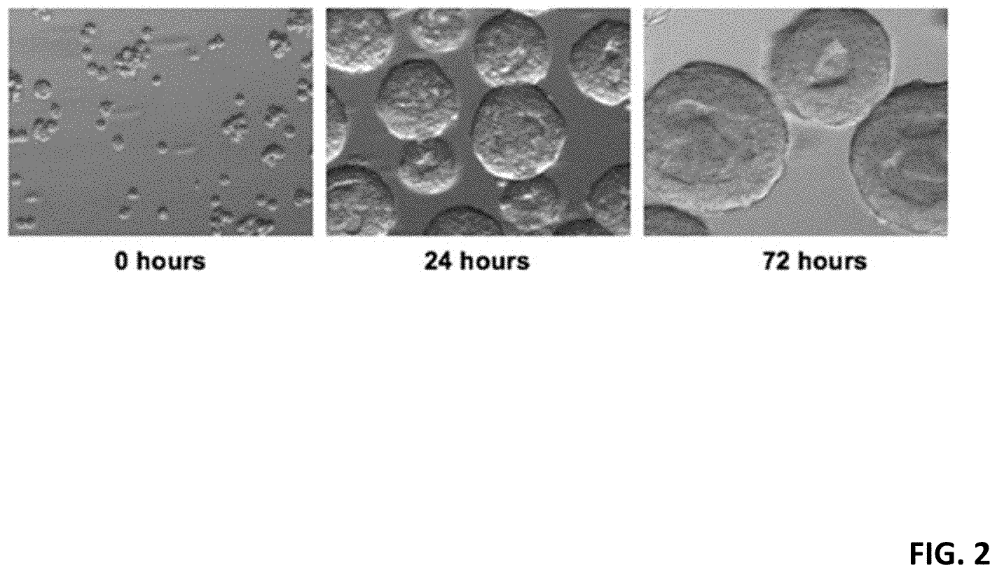

FIG. 2 shows the growth of PSCs as suspended cell aggregates in a vertical-wheel bioreactor.

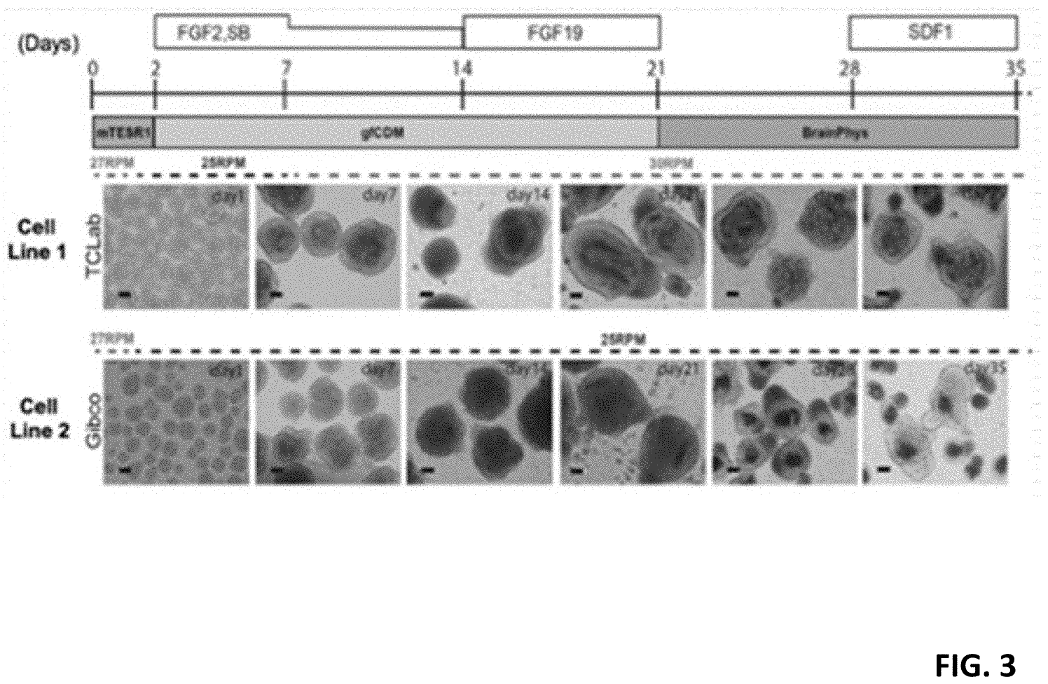

Another characteristic of PSCs is that, after the cell expansion phase, the cell aggregates will typically go through a multi-step process of directed differentiation, to force the pluripotent cells to become a final target therapeutic cell type such as cerebellar cells, which can then form organoids in suspension (FIG. 3).

FIG. 3 show imaging results directed toward the differentiation of human iPSCs to cerebellar organoids in a vertical-wheel bioreactor (FIG. 4) having approximately a 0.1 liter (L) scale. FIG. 3 shows that after 35 days of generation, iPSC-derived organoids were efficiently matured to GABAergic and Glutamatergic neurons (not shown) in PBS-0.1 using a scale bar of approximately 100 micrometers (μm).

Most commercially available microcarriers typically average in diameter between approximately 150 microns to approximately 250 microns (μ), while PSC aggregates of various cell types typically average between approximately 100 microns and approximately 400 microns. Both microcarriers and cell aggregates are substantially uniformly suspended inside a bioreactor to allow the microcarriers to be exposed to the same or similar growth conditions and other biological requirements. Furthermore, these particles are larger than single cells and thus use greater power input to a bioreactor's mixing mechanism, such as an impeller, to be fully and homogenously suspended in culture media. If the mixing environment in a bioreactor is suboptimal or otherwise does not satisfy a threshold level for cells during various process steps, inconsistent yields and poor product quality of cells will occur.

While the biological needs of suspended cells, such as availability of nutrients and removal of waste products are known aspects for achieving a threshold cell culture performance, the fluid mixing environment may also be considered. Therefore, the manner in which a bioreactor suspends and mixes microcarriers or cell aggregates is to be understood and optimized for the mixing environment to remain substantially consistent and substantially predictable during scale up, to allow for large-scale production of therapeutic cell products as is taught based on the teachings of this disclosure.

The teachings of this disclosure generally involve curating the physiological requirements of various types of cell growth techniques, including predicting the threshold fluid dynamic conditions and mixing characteristics of the culture media inside a bioreactor. These parameters are associated with the fluid mixing environment that cells will experience and ultimately affect cell yield and quality throughout a cell culture process.

The teachings of this disclosure also relate to systems and methods, determined by physical mixing studies, power measurements, and computational fluid dynamics (CFD) analyses, for optimum and scalable production of therapeutic cells grown on microcarriers or as cell aggregates in a suspension-based bioreactor. The threshold production conditions can be achieved by monitoring and controlling specific fluid dynamic mixing conditions, which in turn may influence the efficiency of cell expansion for microcarrier-based processes, and both expansion and differentiation processes for PSC aggregate-based processes. For both of these process types, the methods in accordance with the teachings of this disclosure can be used to optimize the yield and quality of final cell products.

The teachings of this disclosure also relate to systems and methods for production of therapeutic cells, including those grown on microcarriers or as cell aggregates, within a bioreactor by controlling fluid dynamic conditions.

Suspension-based cell culture processes in bioreactors may be adapted such that all cells (or substantially are cells) are substantially continuously suspended in the liquid medium. The cells being suspended in the liquid medium ensures or at least enables that the cells may have exposure to a substantially consistent environment of biological and fluid dynamics parameters, and may deter and/or avoid unwanted settling of cells at the bottom of a bioreactor vessel. The agitation rate of a bioreactor's mixing mechanism, such as a rotating impeller, is directly controlled through power input into the impeller.

FIG. 4 illustrates a schematic diagram of an implementation of a system 100 in accordance with the teachings of this disclosure. The system 100 can be used to scale production of therapeutic cells for the biopharmaceutical industry. In the implementation shown, the system 100 includes a first bioreactor 102 including a first containment vessel 104 defining a first working volume 105 and a second bioreactor 106 including a second containment vessel 108 defining a second working volume 107. The first bioreactor 102 may be referred to as a large scale bioreactor and the second bioreactor 106 may be referred to as a small scale bioreactor. As such, as shown, the first working volume 105 is larger than the second working volume 107. However, the first working volume 105 may be smaller than or similar to or the same as the second working volume 107.

In some implementations, the first working volume 105 may be between approximately 250 liters (L) and approximately 500 L, between approximately 45 L and approximately 80 L, between approximately 9 L and approximately 15 L, and/or between approximately 1.8 L and approximately 3.0 L and the second working volume 107 may be between approximately 60 milliliters (mL) and approximately 100 mL and/or between approximately 300 mL and approximately 500 mL. More generally, the first working volume 105 and/or the second working volume 107 may be at least one of between approximately 0.1 L and approximately 500 L or at least one of between approximately 0.0 L and approximately 2000 L. However, the first working volume 105 and/or the second working volume 107 may be any volume.

Referring now to the first bioreactor 102 in detail, in the implementation shown, the first bioreactor 102 includes the containment vessel 104 having walls 110 including a lower curved wall 112 located at a lower end 114 of the containment vessel 104. The first bioreactor 102 also includes a mixer 116 positioned in the containment vessel 104 and configured to rotate about an axis 118 so as to stir contents of the containment vessel 104. The lower curved wall 112 may be referred to as a curved bottom wall and the axis 118 may be referred to as a central axis or a horizontal axis. As shown, the mixer 116 is configured to rotate about a horizontal axis 118. However, the mixer 116 may be differently arranged. For example, the mixer 116 can be configured to rotate about a vertical axis or at an angle relative to the horizontal axis and/or the vertical axis.

The system 100 also includes a drive assembly 122 operatively coupled to the mixer 116 that is adapted to operate/rotate the mixer 116 and a controller 124 having a processor 125. The controller 124 is electrically and/or communicatively coupled to the drive assembly 122 to cause the drive assembly 122 to perform various functions as disclosed herein. The second bioreactor 106 can have similar structures to the first bioreactor 102. For example, the second bioreactor 106 can have the walls 110, the lower curved wall 112, and the mixer 116 having the same or similar dimensions, aspect ratios, and/or bioreactor functions as the first bioreactor 102.

In operation, the second bioreactor 106 is used to perform experiments on a smaller volume to determine values (e.g., agitation rate(s), EDR value(s)) to operate the second bioreactor 106 at to grow therapeutic cells having shapes and/or sizes that are substantially uniform and/or satisfy a threshold standard. Advantageously, based on the operating values at which the second bioreactor 106 is operated, the first bioreactor 106 can determine operating values to operate at to grow therapeutic cells in the larger volume of the first bioreactor 102 having similar or the same desired characteristics of the cells grown in the second bioreactor 106. Put another way, the first bioreactor 102 (the larger-scale bioreactor) is operated based on operating values of the second bioreactor 106 (the smaller-scale bioreactor) to grow therapeutic cells having desired attributes such as, for example, having similar sizes and/or shapes.

In some implementations, at least one of the parameter values includes an agitation rate at which the mixer 116 of the second bioreactor 106 is operated. The agitation rate may be associated with the revolutions per minute (RPMs) at which the mixer 116 of the second bioreactor 106 is rotated. The mixer 116 may be rotated at a rate that achieves an average energy dissipation rate (average EDR) of turbulent eddies within a suspension including the therapeutic cells disposed in the second bioreactor 106. Put another way, the mixer 116 may be rotated at a rate that achieves energy dissipation rates that are distributed throughout the bioreactor volume. The suspension may include microcarriers and the therapeutic cells may include pluripotent stem cells (PSCs). However, the suspension and/or the therapeutic cells may be different. The suspension itself may comprise a liquid media that contains various nutrients, growth factors, chemicals, and/or other additions that are intended to improve growth, differentiation, or other biological performance of cells. The media typically has a similar density to water and while there are commercially available media tailored for specific cell types and process needs, customized media may be produced. Other therapeutic cells types can include, but are not limited to, mesenchymal stem cells or genetically modified single cells such as T-cells. The majority of therapeutic cells are human-derived but may potentially be animal-, insect-, virus-, or bacteria-derived as well.

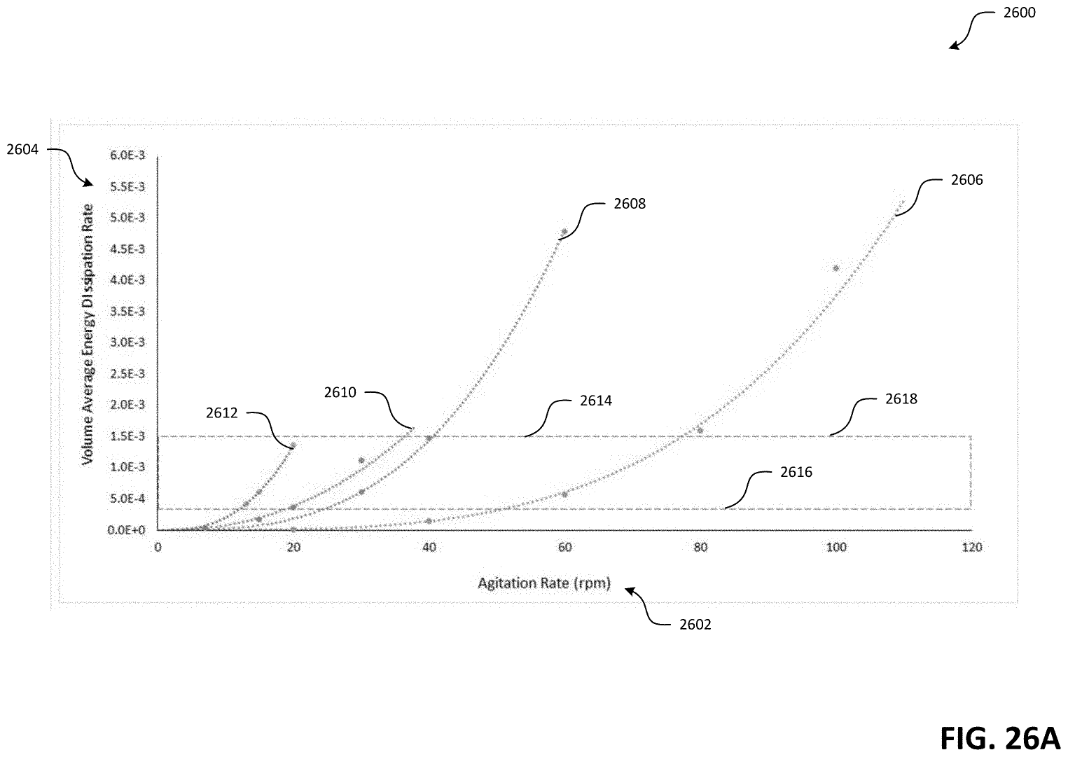

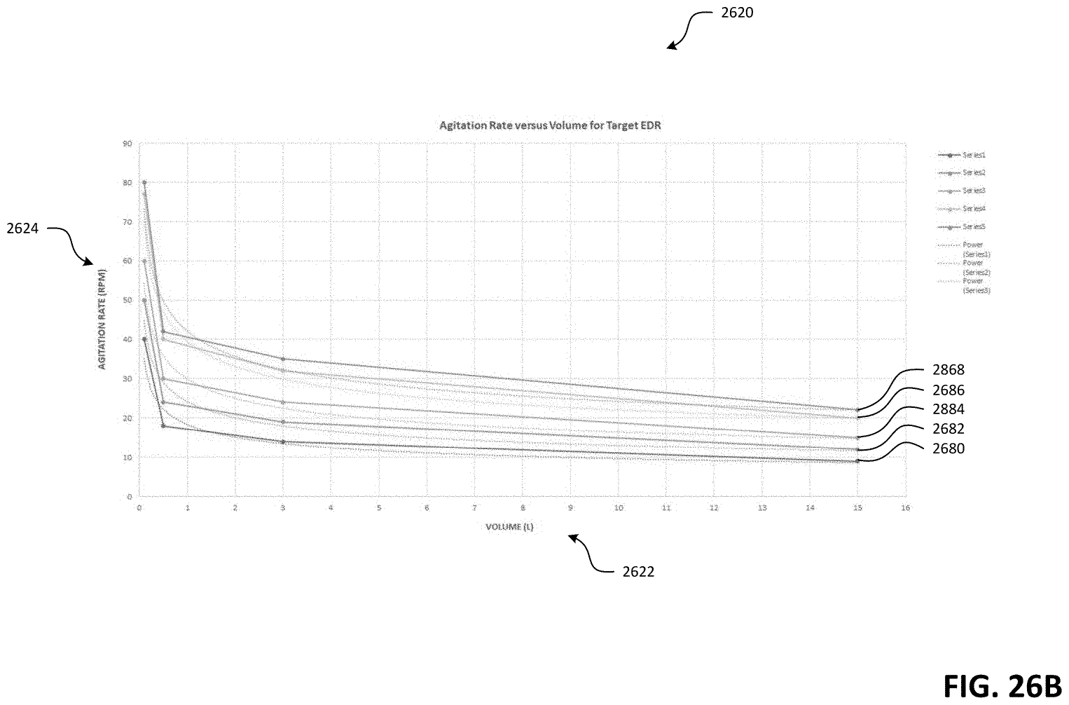

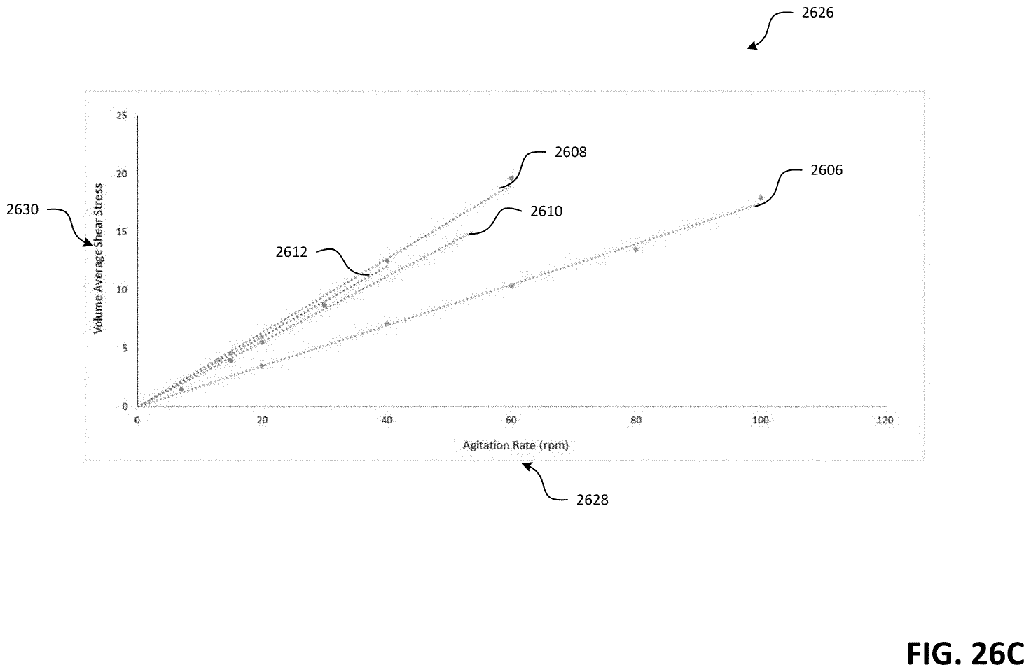

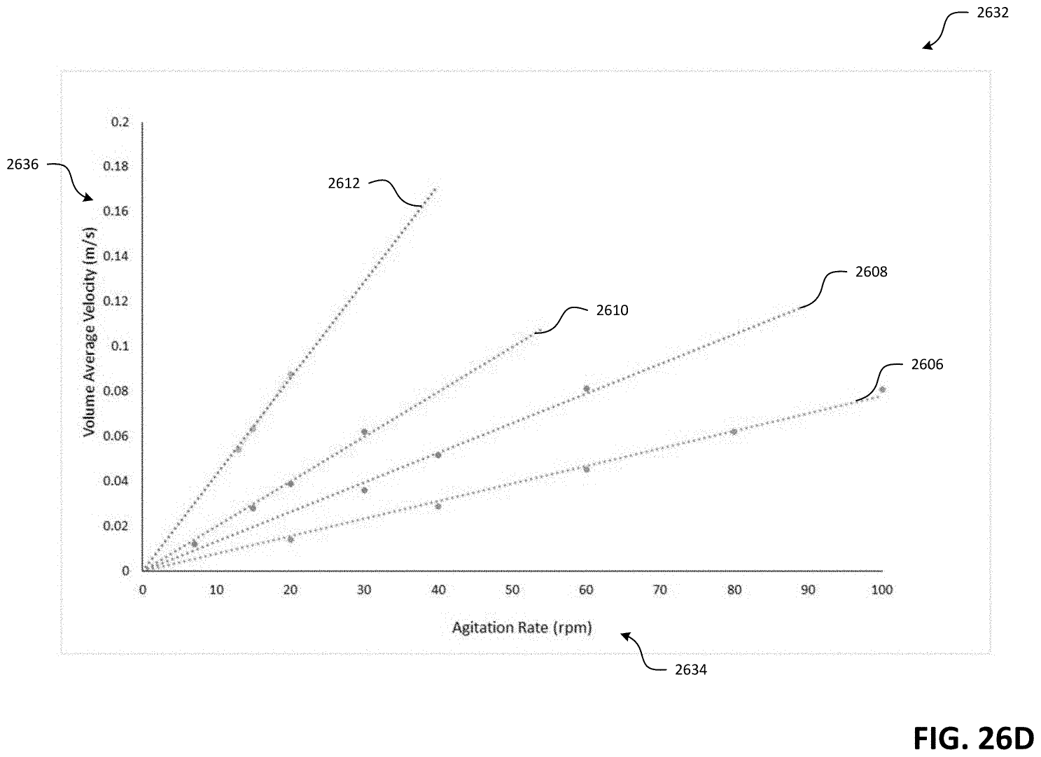

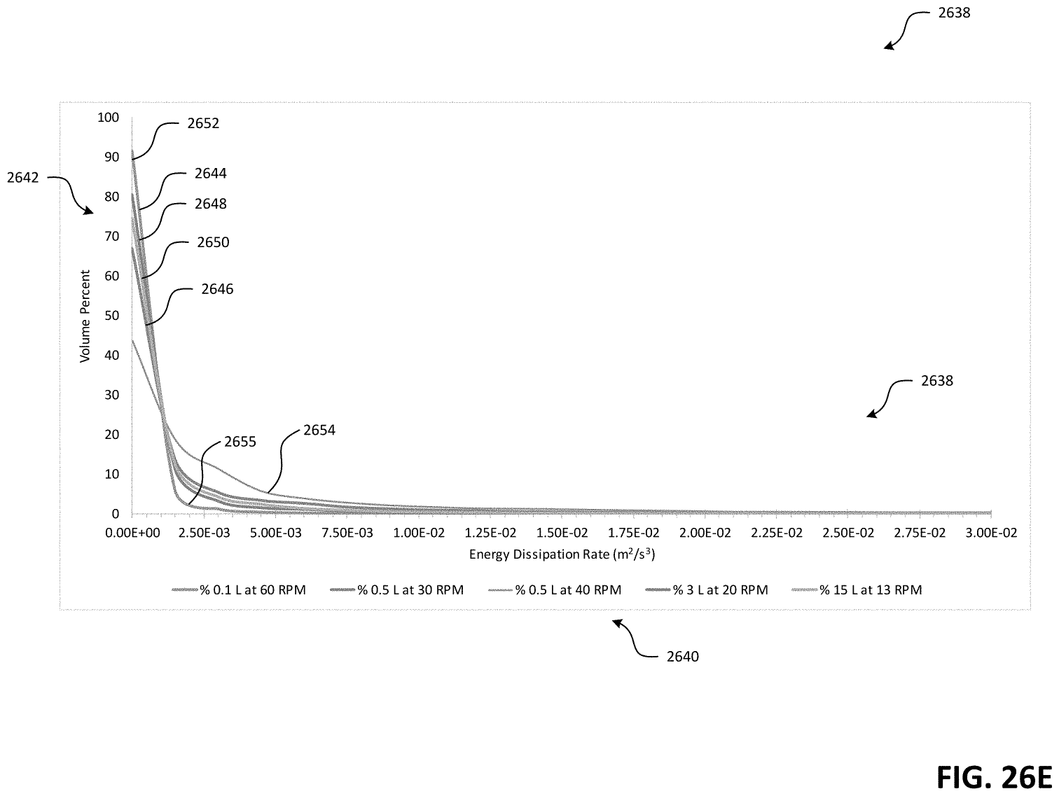

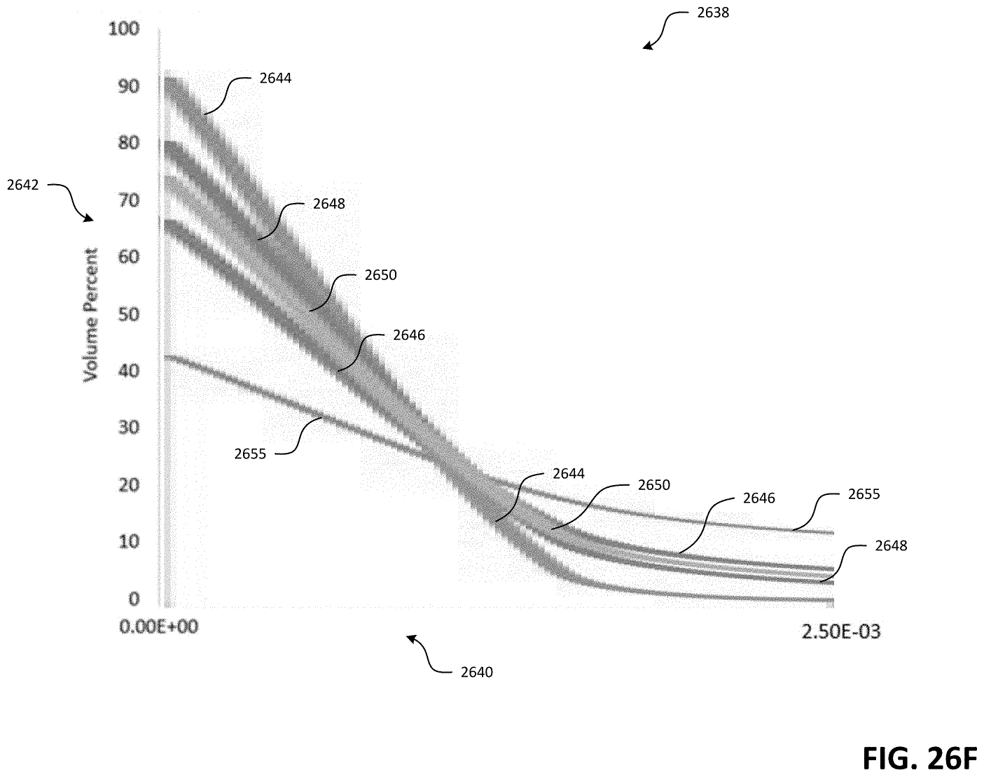

In such implementations, the processor 125 of the controller 124 accesses the agitation rate at which the mixer 116 of the second bioreactor 106 is operated and determines a second agitation rate at which the mixer 116 in the containment vessel 104 of the first bioreactor 102 is to be operated based on the first agitation rate and causes the mixer 116 of the first bioreactor 102 to rotate at the second agitation rate. In other implementations, the agitation rate of the first bioreactor 102 may be determined in a different way. For example, the second agitation rate may be determined manually (e.g., using pen and paper, using a calculator) and/or using a chart or graph (see, FIG. 26A) that associates the first and second agitation rates. The first agitation rate and/or the second agitation rate may be in a range of between approximately 0 revolutions per minute (RPMs) and approximately 120 RPMs and/or between approximately 12 RPMs and approximately 77 RPMs. However, the mixers 116 of the first bioreactor reactor 102 and/or the second bioreactor 106 may be operated at any agitation rate that allow the therapeutic cells produced to satisfy threshold values related to, for example, cells having similar sizes and/or shapes.

The processor 125 may also determine the agitation rate based on additional or alternative inputs. For example, the processor 125 may determine the agitation rate of the first bioreactor 102 based on inputs associated with the media being used, the cell line type, the inoculation condition(s), and a working volume of the first bioreactor 102 and/or the second bioreactor 106. Based on the input value(s) received at or otherwise accessed by the processor 125, the processor 125 may access a data base, such as the memory 138 of the controller 124, and compare the input(s) received to reference data (e.g., historical data) stored in the memory 138. The reference data may contain data from experiments performed at other bioreactors using different media(s), different agitation rate(s), different cell line type(s), different inoculation condition(s), and/or different working volume(s) and may accessed by the controller 124 using, for example, the communication interface 136. Advantageously, in such an example, the processor 125 can compare the received input value(s) to the reference data and the processor 125 can then determine an agitation rate to operate the first bioreactor 102 at that will more likely grow cells having similar sizes and/or shapes that is tailored to the particular conditions (e.g., working volume, cell line type, media). Put another way and as an example, the processor 125 dynamically provides first feedback to a user to operate the first bioreactor 102 at a first agitation rate if a first working volume is to be used with the first bioreactor 102 and the processor 125 dynamically provides second feedback to a user to operate the first bioreactor 102 at a second agitation rate if a second working volume is to be used with the first bioreactor 102.

Operating the second bioreactor 106 at the first agitation rate achieves an average energy dissipation rate (average EDR) of turbulent eddies within a suspension including cells disposed in the second bioreactor 106 and operating the mixer 116 of the first bioreactor 102 at the second agitation rate substantially achieves a target average EDR of turbulent eddies within a suspension including cells in the containment vessel 104. In some implementations, the target average EDR is approximately equal to the average EDR. The turbulent eddies generated by operating the mixer 116 at the second agitation rate. A magnitude of the EDR of at least approximately 60%, at least approximately 80%, approximately 85%, approximately 90%, approximately 95%, or approximately 97% of the turbulent eddies is less than approximately 0.0015 m²/s³. As set forth herein, approximately 0.0015 m²/s³ is +/−50% or equal to 0.0015 m²/s³.

In some implementations, the target average EDR is in a range of between approximately 0 m²/s³ and approximately 0.006 m²/s³. In other implementations, the target average EDR is in a range of between approximately 0.0003 m²/s³ and approximately 0.0015 m²/s³. However, different target average EDRs may be suitable depending on, for example, the therapeutic cells being produced, the volume of the containment vessel 104, 108, etc.

The dissipation of the kinetic energy of turbulence (the energy associated with turbulent eddies in a fluid flow) is the rate at which the turbulence energy is absorbed by breaking the eddies down into smaller and smaller eddies until the eddy is ultimately converted into heat by viscous forces. EDR may be expressed as the kinetic energy per unit mass per second, with units of velocity squared per second (m²/s³). A narrow range of EDR may be associated with a homogeneous fluid flow environment with eddies that do not vary substantially in size. Sufficiently small Kolmogorov eddies can also have a physical shearing effect on cells attached.

Still referring to FIG. 4, in the implementation shown, the first bioreactor 102 is a vertical-wheel bioreactor 102 that is used as a mixing mechanism to create a substantially uniform mixing environment within the containment vessel 104. The drive assembly 122 includes a drive mechanism 126 such as a magnetic drive 127 about which the wheel 120 rotates to create tangential fluid flow around a circumference 128 of the wheel 120. The magnetic drive 127 may be referred to as a magnetic coupling.

The wheel 120 includes oppositely-oriented axial vanes 130 that may create a cutting-and-folding action of the fluid through the axis 118 and may provide relatively efficient mixing at relatively low power inputs to, for example, the drive assembly 122 and/or the wheel 120. Moreover, in the implementation shown, the wheel 120 includes an impeller zone 132 that is sized to produce a relatively low energy dissipation rate (EDR) and gentle mixing. For example, the impeller zone 132 may be relatively large to produce a relatively large swept volume. The wheel 120 and the containment vessel 104 operate together to create strong and sweeping flow that can fully suspend large particles, such as plastic microcarriers or cell aggregates within the containment vessel 104, with relatively low power input as compared to traditional stirred-type bioreactors (STRs) with horizontal-impeller mixing.

In other implementations, the drive assembly 122 can be omitted and the containment vessel 104 may include an air-input port (not shown) that flows air into the container vessel 104 to create buoyant air bubbles that rise, interact with the wheel 120 and pneumatically turn the wheel 120. Rotating the wheel 120 using air bubbles may result in the same or similar fluid flow characteristics sufficient for low-power suspension of microcarriers or cell aggregates similar to using the drive assembly 122 disclosed above. However, the action of air bubbles popping at a liquid surface within the containment vessel 104 is a potential source of shear damage to anchorage-dependent cells and, thus, magnetic drive mixing is preferred for cell types such as MSCs or PSCs.

In operation, as the mixer 116 of the first bioreactor 102 and/or the second bioreactor 106 rotates in the suspension contained within the containment vessel 104, 108, the mixer 116 creates turbulent flow that includes Kolmogorov eddies of various sizes. Larger eddies break down into smaller and smaller eddies due to viscous forces, until the smallest eddies dissipate and are converted into heat. EDR is the rate of this energy loss as eddies are converted from kinetic energy to thermal energy and a narrow range of EDR is associated with a homogeneous fluid flow environment with eddies that do not vary widely in size. Sufficiently small Kolmogorov eddies can also have a physical shearing effect on cells attached to the surface of the microcarriers. In the context of microcarrier-based processes, shear forces can potentially have a detrimental impact on cells attached to the surface of suspended microcarriers.

While the EDR is mentioned above as affecting the mixing environment within the containment vessel 104, other parameters may be relevant. For example, some of these parameters include: minimal power input to the impeller, a substantially homogeneous energy dissipation rate (EDR), and relatively low hydrodynamic shear stress levels. Changes to power input to the mixer 116 from the drive assembly 122 change the agitation rate within the containment vessel 104 and directly affect both the levels of EDR and shear stress. EDR may scale exponentially with increased agitation while shear stress scales linearly.

When the bioreactors 102 and/or 106 are operated in a manner that creates eddies that are larger than the diameter of a suspended microcarrier, the wave-like eddy streamlines sweep the microcarriers with the attached cells along the fluid flow path. In contrast, when the bioreactors 102 and/or 106 are operated in a manner that creates eddies that become significantly smaller than the diameter of a microcarriers, the smaller eddies create a shearing effect to the cells on the surface of microcarriers that causes cell damage or even death of the cells. Thus, power input to the wheel 120 using the drive assembly 122 directly affects eddy size inside the vessels 104 and/or 108 when the mixer 116 is rotated at a faster rate. A higher power input generates a mixing action that creates smaller eddies.

Referring to the controller 124, in the implementation shown, the controller 124 includes a user interface 134, a communication interface 136, one or more processors 125, and a memory 138 storing instructions executable by the one or more processors 125 to perform various functions including the disclosed implementations. The user interface 134, the communication interface 136, and the memory 138 are electrically and/or communicatively coupled to the one or more processors 125.

In an implementation, the user interface 134 is adapted to receive input from a user and to provide information to the user associated with the operation of the system 100 and/or an analysis taking place. The input may include, for example, a first agitation rate value at which the second bioreactor 106 is operated, an average EDR value achieved by operating the second bioreactor 106 at the first agitation rate value, a second agitation rate value at which the first bioreactor 102 is to be operated, and/or an average EDR rate value achieved by the first bioreactor 102 being operated at the second agitation rate value. However, the user interface 134 or, more generally, the controller 124 may receive other inputs. Some of these inputs may be associated with providing minimal power input to the mixer 116 and/or the drive assembly 122, achieving a substantially homogeneous energy dissipation rate (EDR), and/or achieving relatively low hydrodynamic shear stress levels. Additionally or alternatively, the input(s) may include, for example, the media, the cell line type, an inoculation condition, a working volume of the bioreactor 102, 106. The user interface 134 may include a touch screen, a display, a key board, a speaker(s), a mouse, a track ball, and/or a voice recognition system. The touch screen and/or the display may display a graphical user interface (GUI).

In an implementation, the communication interface 136 is adapted to enable communication between the first bioreactor 102 and the second bioreactor 106 and/or a remote system(s) (e.g., computers) via a network(s). The network(s) may include the Internet, an intranet, a local-area network (LAN), a wide-area network (WAN), a coaxial-cable network, a wireless network, a wired network, a satellite network, a digital subscriber line (DSL) network, a cellular network, a Bluetooth connection, a near field communication (NFC) connection, etc. Some of the communications provided to the remote system may be associated with analysis results, etc. generated or otherwise obtained by the first bioreactor 102. Some of the communications provided to the first bioreactor 102 may be associated with a mixing operation to be executed by the first bioreactor 102 and/or an agitation rate, an average EDR, and/or a target average EDR.

The one or more processors 125 and/or the system 100 may include one or more of a processor-based system(s) or a microprocessor-based system(s). In some implementations, the one or more processors 125 and/or the system 100 includes one or more of a programmable processor, a programmable controller, a microprocessor, a microcontroller, a graphics processing unit (GPU), a digital signal processor (DSP), a reduced-instruction set computer (RISC), an application specific integrated circuit (ASIC), a field programmable gate array (FPGA), a field programmable logic device (FPLD), a logic circuit and/or another logic-based device executing various functions including the ones described herein.

The memory 138 can include one or more of a semiconductor memory, a magnetically readable memory, an optical memory, a hard disk drive (HDD), an optical storage drive, a solid-state storage device, a solid-state drive (SSD), a flash memory, a read-only memory (ROM), erasable programmable read-only memory (EPROM), electrically erasable programmable read-only memory (EEPROM), a random-access memory (RAM), a non-volatile RAM (NVRAM) memory, a compact disc (CD), a compact disc read-only memory (CD-ROM), a digital versatile disk (DVD), a Blu-ray disk, a redundant array of independent disks (RAID) system, a cache and/or any other storage device or storage disk in which information is stored for any duration (e.g., permanently, temporarily, for extended periods of time, for buffering, for caching).

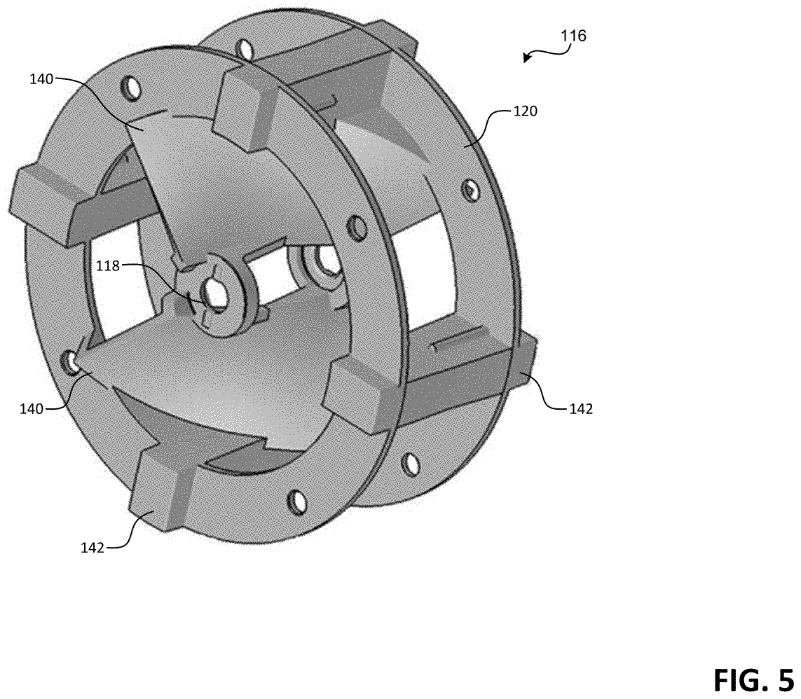

FIG. 5 is an isometric view of the mixer 116 that can be used with the bioreactors 102, 106 of FIG. 4. In the implementation shown, the mixer 116 includes the wheel 120 including two axial-flow vanes 140 and four radial-flow blades 142. While the mixer 116 includes two axial-flow vanes 140 and four radial-flow blades 142, any number of axial-flow blades may be included (e.g., 1, 3, 4) and/or any number of radial-flow blades 142 may be included (e.g., 1, 2, 3). In other implementations, the axial-flow vanes 140 and/or the radial flow blades 142 may be omitted.

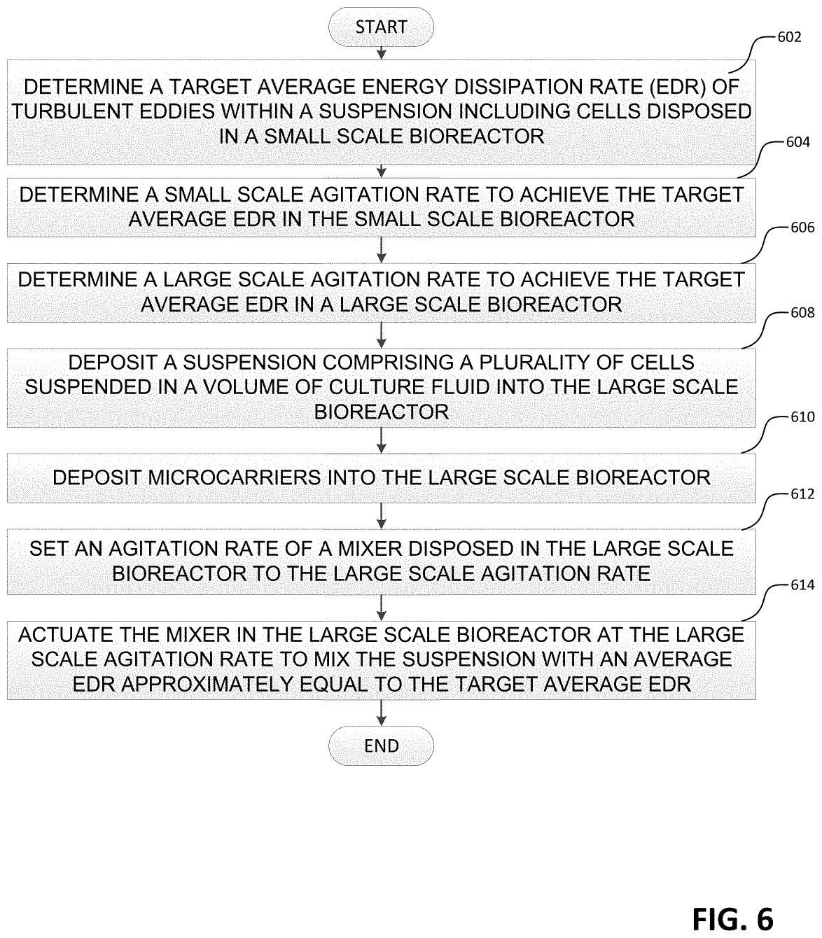

FIGS. 6-8 illustrate flowcharts for methods of scaling production of therapeutic cells grown on microcarriers or as cell aggregates in a suspension-based bioreactor using the system 100, the first bioreactor 102, and/or the second bioreactor 106 of FIG. 4 or any of the other implementations disclosed herein. The order of execution of the blocks may be changed, and/or some of the blocks described may be changed, eliminated, combined and/or subdivided into multiple blocks.

The process 600 of FIG. 6 begins with a target average energy dissipation rate (EDR) of turbulent eddies within a suspension including cells disposed in a small scale bioreactor 106 being determined (Block 602). In some implementations, the target average EDR is determined using the processor 125, manually (e.g., pen and paper), using another computer, or by referring a chart or graph (see, FIG. 26A). The target average EDR may be in a range of between approximately 0 m²/s³ and approximately 0.006 m²/s³ and/or in a range of between approximately 0.0003 m²/s³ and approximately 0.0015 m²/s³. A small scale agitation rate is determined that achieves the target average EDR in the small scale bioreactor 106 (Block 604) and a large scale agitation rate is determined that achieves the target average EDR in a large scale bioreactor 102 (Block 606). The large scale agitation rate is directly dependent on the small scale agitation rate. The large scale bioreactor 102 has a volume larger than a volume of the small scale bioreactor 106 and, in some implementations, at least one of the small scale and large scale agitation rates is in a range of between approximately 0 rpm and approximately 120 rpm and/or in a range of between approximately 12 rpm and approximately 77 rpm.

A suspension including a plurality of cells suspended in a volume of culture fluid is deposited into the large scale bioreactor 102 (Block 608). Depositing the suspension including the cells into the large scale bioreactor 102 may include depositing pluripotent stem cells (PSCs) or mesenchymal stem cells (MSCs) into the large scale bioreactor 102. Microcarriers are optionally deposited, for example in combination with MSCs, into the large scale bioreactor (Block 610). The microcarriers and the suspension may be deposited in the large scale bioreactor 102 at the same time, different times in sequence, and/or at similar times (e.g., one after the other and/or within a time period).

The agitation rate of the mixer 115 disposed in the large scale bioreactor 102 is set to the large scale agitation rate (Block 612) and the mixer 116 in the large scale bioreactor 102 is actuated at the large scale agitation rate to mix the suspension with an average EDR approximately equal to the target average EDR (Block 614). The average EDR may include an average of a plurality of actual EDR data points within the volume of the suspension in the large scale bioreactor 102, where a magnitude of at least approximately 60%, at least approximately 80%, approximately 85%, approximately 90%, approximately 95%, or approximately 97% of the plurality of actual EDR data points is less than approximately 0.0015 m²/s³.

In some implementations, actuating the mixer 116 in the large scale bioreactor 102 includes actuating a vertical wheel mixer 116 having a horizontal axis 118 of rotation. In other implementations, actuating the mixer 116 in the large scale bioreactor 102 includes actuating the mixer 116 having a vertical axis of rotation such as a spinner type mixer. However, the mixer 116 may be differently configured and/or located within the containment vessel 104, for example, and may include different means for agitation including pneumatics (e.g., a bubble mixer) and/or other mechanisms.

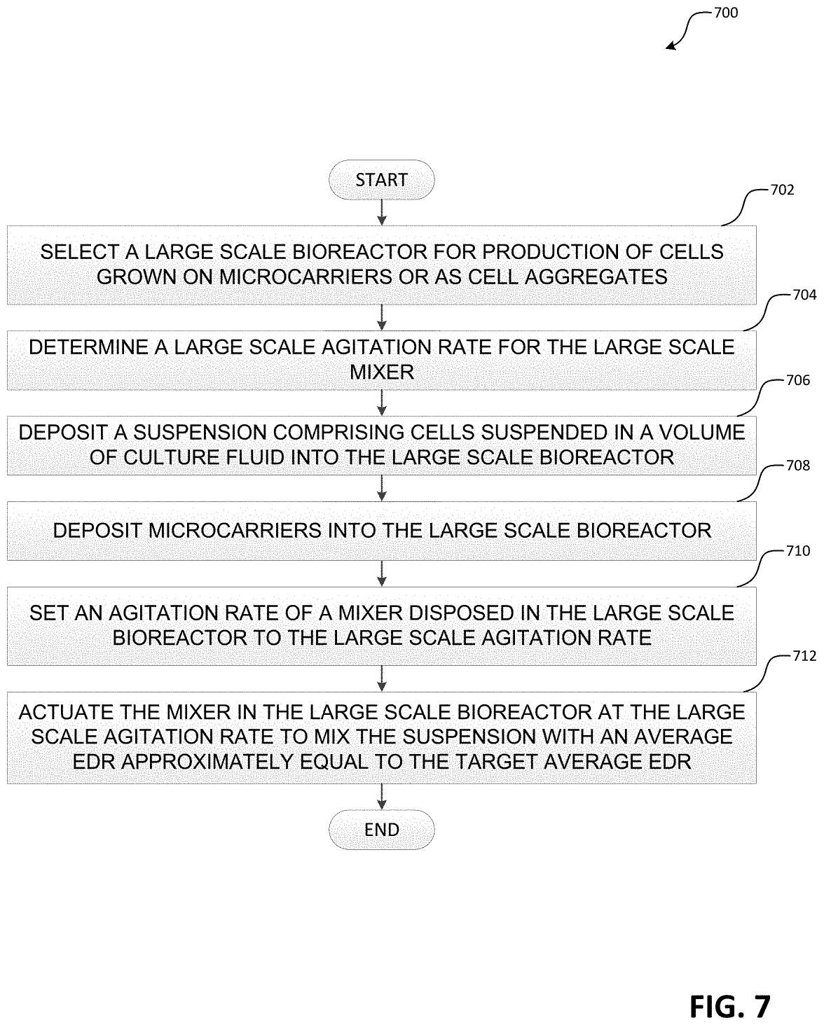

The process 700 of FIG. 7 begins with a large scale bioreactor 102 being selected for production of cells grown on microcarriers or as cell aggregates (Block 702). Selecting the large scale bioreactor 102 includes selecting a bioreactor 102 with a volume larger than a volume of the small scale bioreactor 106. The large scale bioreactor 102 has a large scale mixer 116 in a large scale vessel 104. A large scale agitation rate is determined for the large scale mixer 116. (Block 704) The large scale agitation rate is determined based on a small scale agitation rate of a small scale mixer 116 in a small scale vessel 108 of a small scale bioreactor 106 that achieves a target average energy dissipation rate (EDR) of turbulent eddies in a suspension in the small scale bioreactor 106. In some implementations, at least one of the small scale and large scale agitation rates is in a range of between approximately 0 rpm and approximately 120 rpm and/or in a range of between approximately 12 rpm and approximately 77 rpm. In some implementations, the target average EDR is in a range of between approximately 0 m²/s³ and approximately 0.006 m²/s³ and/or in a range of between approximately 0.003 m²/s³ and approximately 0.0015 m²/s³.

A suspension including cells suspended in a volume of culture fluid is deposited into the large scale bioreactor 102 (Block 706). Depositing the suspension including cells into the large scale bioreactor may include depositing pluripotent stem cells (PSCs) or mesenchymal stem cells (MSCs) into the large scale bioreactor. Microcarriers can then optionally be deposited into the large scale bioreactor (Block 708), for example, when used with MSCs. The microcarriers and the suspension may be deposited in the large scale bioreactor 102 at the same time, different times in sequence, and/or at similar times (e.g., one after the other and/or within a time period).

The agitation rate of the large scale mixer 116 is set to the large scale agitation rate (Block 710) and the large scale mixer 116 is actuated at the large scale agitation rate to mix the cells in the suspension at an average EDR approximately equal to the target average EDR (Block 712). In some implementations, the average EDR includes an average of a plurality of actual EDR data points within the volume of the suspension in the large scale bioreactor 102, where a magnitude of at least approximately 60%, at least approximately 80%, approximately 85%, approximately 90%, approximately 95%, or approximately 97% of the plurality of actual EDR data points is less than approximately 0.0015 m²/s³. In some implementations, actuating the large scale mixer 116 includes including a vertical wheel mixer 116 having a horizontal axis 118 of rotation. In other implementations, actuating the large scale mixer 116 includes actuating a mixer 116 having a vertical axis of rotation such as a spinner type mixer. Other mixers are possible and may include different means for agitation including pneumatics (e.g., a bubble mixer) and/or other mechanisms.

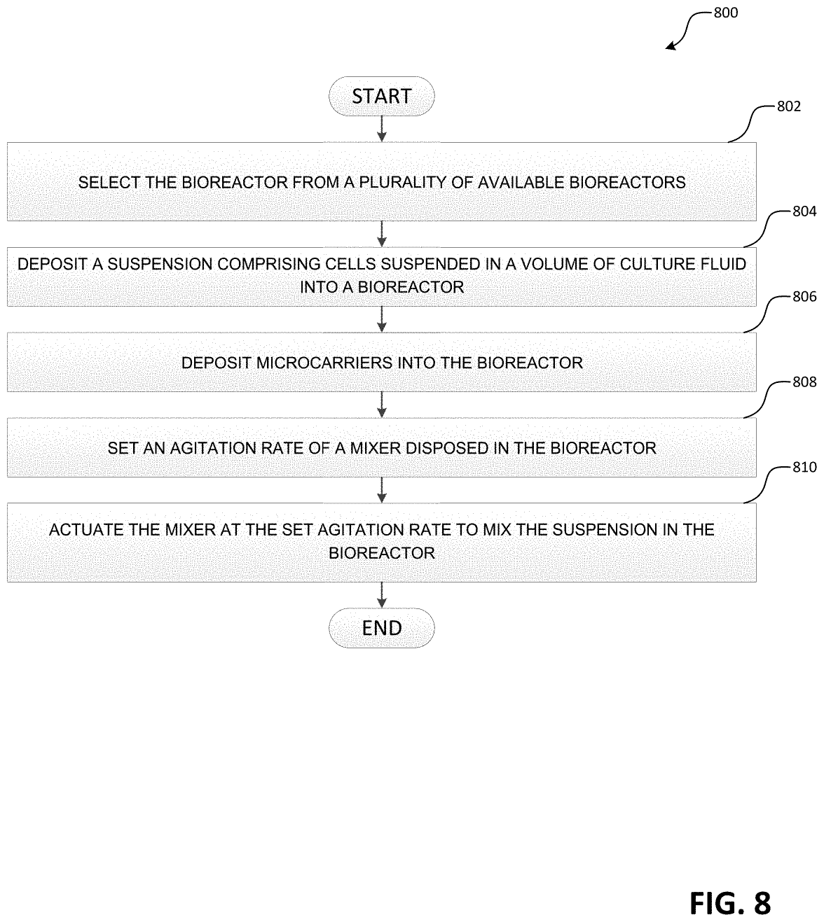

The process 8 of FIG. 8 begins with the bioreactor 102, 106 being selected from a plurality of available bioreactors 102, 106 (Block 802). Each of the bioreactors 102, 106 has a volume of between approximately 0.1 L and approximately 500 L or between approximately 0.1 L and approximately 2000 L. The bioreactor 102, 106 includes the mixer 116. The mixer 116 may be a vertical wheel mixer 116 having a horizontal axis 118 of rotation or may have a vertical axis of rotation. The bioreactor 102, 106 includes a containment vessel 104, 108 that may have the curved bottom wall 112.

A suspension comprising cells suspended in a volume of culture fluid is deposited into a bioreactor (Block 804). Depositing the suspension including cells suspended in a volume of a culture fluid into the bioreactor may include depositing pluripotent stem cells (PSCs) or mesenchymal stem cells (MSCs) into the bioreactor 102. Microcarriers can then be optionally deposited, for example with the MSCs, into the bioreactor (Block 806) and an agitation rate of the mixer 116 disposed in the bioreactor 102 is set (Block 808). The mixer 116 is actuated at the set agitation rate to mix the suspension in the bioreactor (Block 810). The suspension includes turbulent eddies generated by the mixer and the turbulent eddies each having an energy dissipation rate (EDR). A magnitude of the EDR of at least approximately 60%, at least approximately 80%, approximately 85%, approximately 90%, approximately 95%, or approximately 97% of the turbulent eddies is less than approximately 0.0015 m²/s³. In some implementations, the target average EDR is in a range of between approximately 0 m²/s³ and approximately 0.006 m²/s³ and/or in a range of between approximately 0.0003 m²/s³ and approximately 0.0015 m²/s³.

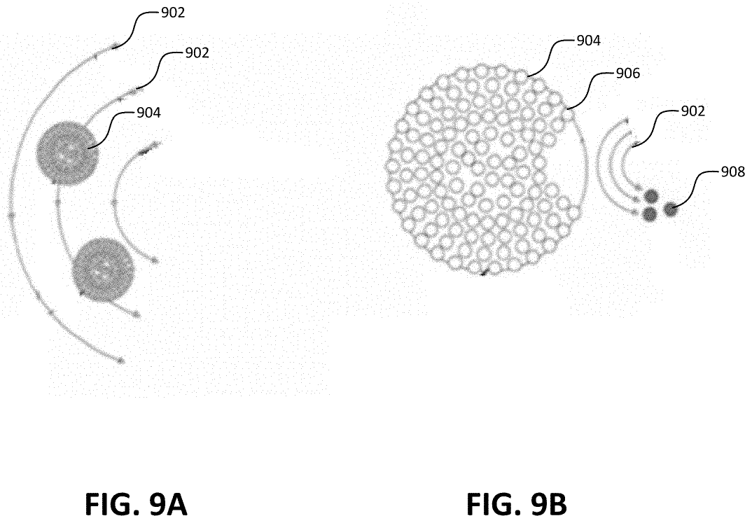

FIG. 9A illustrates a schematic representation of eddy streams 902 and microcarriers 904 with attached viable cells when the eddy streams 902 are larger than the microcarriers 904. FIG. 9B illustrates a schematic representation of one of the microcarriers 904 with attached viable cells 906 and eddy streams 902 that are smaller than the microcarriers 904. As shown in FIG. 9B, some dead cells 908 have detached.

Referring to FIG. 9A, when eddies 902 are larger than the diameter of a suspended microcarrier 904, the wave-like eddy streamlines 902 sweep the entire microcarriers 904 with attached cells along the fluid flow path. In contrast and with reference to FIG. 9B, when eddies 902 become significantly smaller than the diameter of a microcarriers 904, there will be a shearing effect to the cells on the surface of the microcarriers 904, which may cause cell damage or even death. Power input to a bioreactor's impeller 116 directly affects eddy size inside the vessel 104 and higher power input for faster rotation or mixing action will create smaller eddies.

While homogenous EDR is beneficial for even distribution of microcarriers and nutrients in liquid, another factor for microcarrier-based cell culture processes is maintaining shear stress levels below a threshold that would damage the cells. An impeller design that promotes formation of larger eddies may be beneficial if shear forces are of particular interest, but minimizing power input to the impeller 120 while still achieving full, off-bottom suspension of microcarriers may be desirable.

In contrast, a factor for aggregate formation of cells such as PSCs is a uniform mixing environment, which is created mainly through homogeneous distribution of all or at least some EDR values with narrow variation. This promotes the formation of spherical cell aggregates having uniform shape and size, which is often helpful for biological requirements during both expansion and differentiation process steps. Adjusting power input and therefore agitation rate, along with maintaining relatively consistent shear stress levels, also has a direct impact on controlling the size of cell aggregates.

As has been shown via studies with microcarriers, the agitation power used for complete suspension of such particles depends strongly on the agitator and bioreactor geometry. The power used for complete suspension depends upon agitation and bioreactor geometry and microcarrier diameter and the density difference between the microcarrier and culture fluid. A typical microcarrier has a diameter of between approximately 150 and approximately 200 microns and specific density of between approximately 1.03 and approximately 1.04, resulting in a density difference of 0.03 to 0.04 g/ml higher than the culture fluid.

A typical PSC aggregate has a diameter of between approximately 100 and approximately 400 microns. The aggregates are comprised of cells which can have different specific densities, as follows, depending upon the cell type: 1.05-1.15 for hepatocytes, 1.04-1.08 for Hela cells, 1.03-1.05 for fibroblasts, and 0.92 for fat cells. Based on these densities, PSC aggregates can be considered to have similar diameters and densities as microcarriers typically used for suspension cell culture processes. Thus, for a given bioreactor, the power input and agitation rate that successfully suspends microcarriers will also work well for suspension of most cell aggregates.

To determine the agitation power to be used for microcarrier suspension in a vertical-wheel bioreactor, the relationship between power number and Reynolds number for the vertical wheel system was characterized. The Reynolds number is used to determine whether fluid flow is laminar or turbulent and can predict the pattern of fluid flow. Characterizing the relationship between the power number and the Reynolds number is the well-established approach that has been used many times for horizontal impeller systems. In some implementations, the second bioreactor 106 was used that has a 0.5 L vertical-wheel single-use vessel. The wheel 120 of the second bioreactor 106 may have a diameter of approximately 7.24 centimeters (cm) and the lower curved wall 112 may have a radius of approximately 4.25 cm. However, different sized wheels 120 and/or lower curved walls 112 may be used.

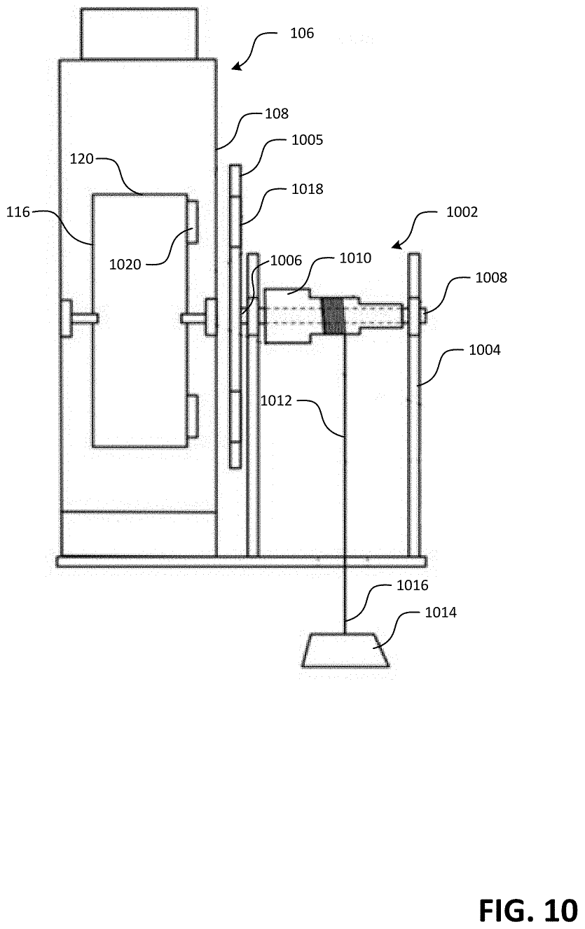

FIG. 10 is a schematic illustration of the second bioreactor 106 and a jig 1002 coupled to the second bioreactor 106 and configured to measure impeller power inputs. In the implementation shown, the jig 1002 includes a base 1004 and a magnetic drive wheel 1005 mounted on a shaft/axle 1006 supported by ball bearings 1008. In the implementation shown, a hollow spindle 1010 is fitted over the shaft 1006 and fine thread 1012 is wound in a single layer on the spindle 1010. A small box 1014 is attached to a loose end 1016 of the thread 1012. The jig 1002 can be installed approximately 2.2 meters (m) above the ground.

The second bioreactor 106 is shown mounted next to the magnetic drive wheel 1005 so that drive magnets 1018 and vessel impeller magnets 1020 may be coupled (magnetically coupled). A Hall Effect sensor connected to a digital tachometer can be used to measure agitation. Known weights may be placed in the box 1014 and the box 1014 can be allowed to fall under the influence of gravity. The total friction of the system (fluid drag+friction of the jig bearings+friction of the wheel bearings) arrests the acceleration of the box 1014 and the box 1014 falls at approximately a constant (terminal) velocity. The velocity of the box 1014 is directly related to the agitation through the circumference of the spindle 1010. The total power to drive the system is equal to the product of the velocity of the falling mass, the mass itself, and the gravitational acceleration constant, g.

To perform experiments using the second bioreactor 106 and/or the jig 1002, power inputs to the agitator (the wheel 120) where carefully measured. Traditional measurements using a dynamometer or transmitting torque meter were deemed impractical due to both the very low power levels involved and the magnetically coupled nature of the wheel 120 rotating. Others have noted these measurement challenges under low power scenarios. Furthermore, in this case, there is no vertical shaft running through the system to which to attach traditional instrumentation. Accordingly, a “gravimetric” approach was settled on.

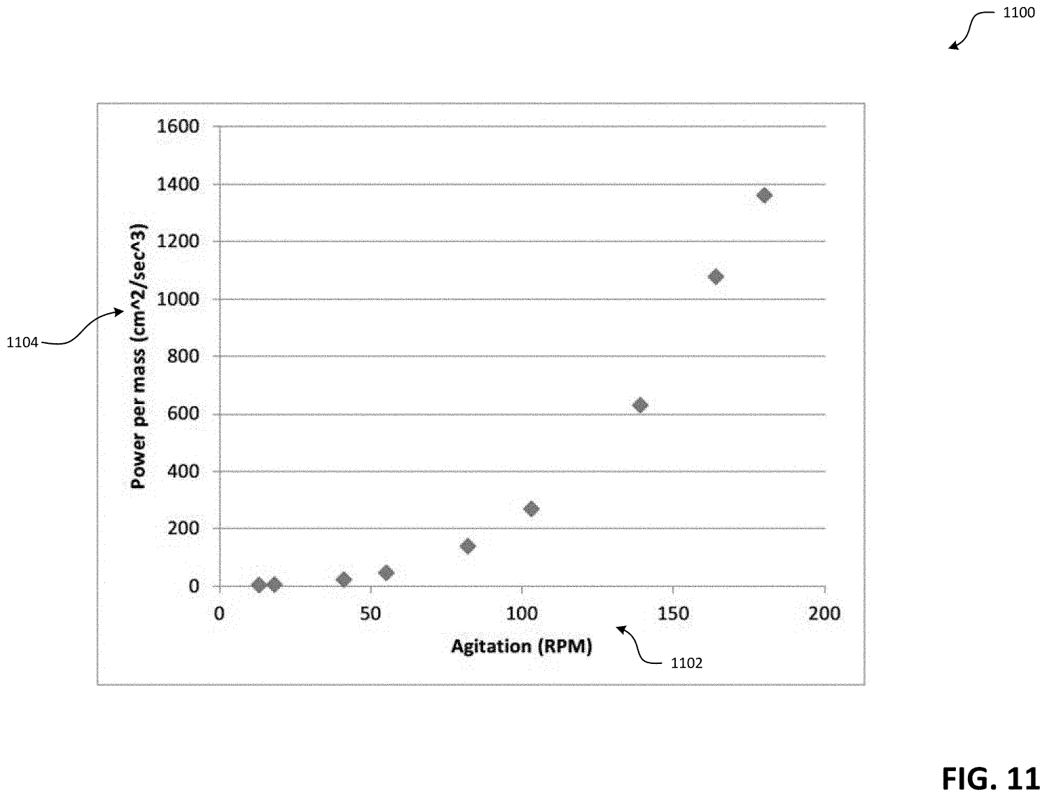

FIG. 11 is a graph 1100 including an X-axis 1102 representing agitation of the wheel 120 of the second bioreactor 106 and a Y-axis 1104 representing power per mass. More specifically, FIG. 11 shows impeller power input per mass as a function of agitation for the second bioreactor 106 having a volume of 0.5 L and a working volume of 300-ml with deionized (DI) water at approximately 26° C. As shown in the graph 1100, when the mixer 116 is rotated at a higher agitation rate, the power per mass rate also increases. At 13 rpm, the lowest speed tested, the power input per mass was approximately 4.1 cm²/sec³ and, at 180 rpm, the highest speed tested, the power input per mass was approximately 1360 cm²/sec³.

To obtain the data plotted on the graph 1100, measurements were done with the jig 1002 alone to measure the dynamic friction of the jig bearings 1008, with the jig 1002 and the containment vessel 108 empty (to measure the dynamic friction of the jig bearings 1008+the vessel bearings), and with the containment vessel 108 containing water (to measure both of the bearing's friction+impeller drag.) Each measurement was done with multiple weights, and the power versus agitation curves were plotted on the graph 1100. Power or polynomial curve fits were done for the condition when the containment vessel 108 was empty, and these fits were used to interpolate the power for any agitation rate or at least a number of agitation rates. This data was used to subtract the friction when the containment vessel 108 was empty from the total system friction (measured with the containment vessel 108 containing water) to obtain the net power. The net power is equal to the impeller drag or to the net mixing power. These experiments were conducted at approximately 26 degree Celsius using water without microcarriers. Standard equations for un-gassed Newtonian fluids without solids were used to determine impeller Reynolds number and power number. The density and viscosity of the water were assumed to be 1.00 g/ml and 0.0085 g/(cm-sec), respectively.

The measurements performed with the jig 1002 and described above were validated by testing it with a standard impeller/bioreactor geometry, wherein the relationship between power number and Reynolds number is well established. The experimental results could then be compared against the well-established ones (i.e., expected results). The standard impeller/bioreactor geometry chosen was a standard baffled stirred tank with a standard (horizontal) Rushton impeller. For this standard geometry operated in the turbulent regime, the expected power number is 5-6 and the expected exponent on a graph of Log P versus Log N expected is 3, wherein P is the power and N is the agitation rate. Using the measurement system described above, rotated 90 degrees to work with this standard geometry operated in the turbulent regime, the power number measured was 5.55, in the middle of the expected 5-6 range, and the log P vs. log N exponent measured was 3.1, quite close to the expected number of 3. Thus, the measurement system was considered validated and used to measure power for the 0.5 L vertical-wheel bioreactor.

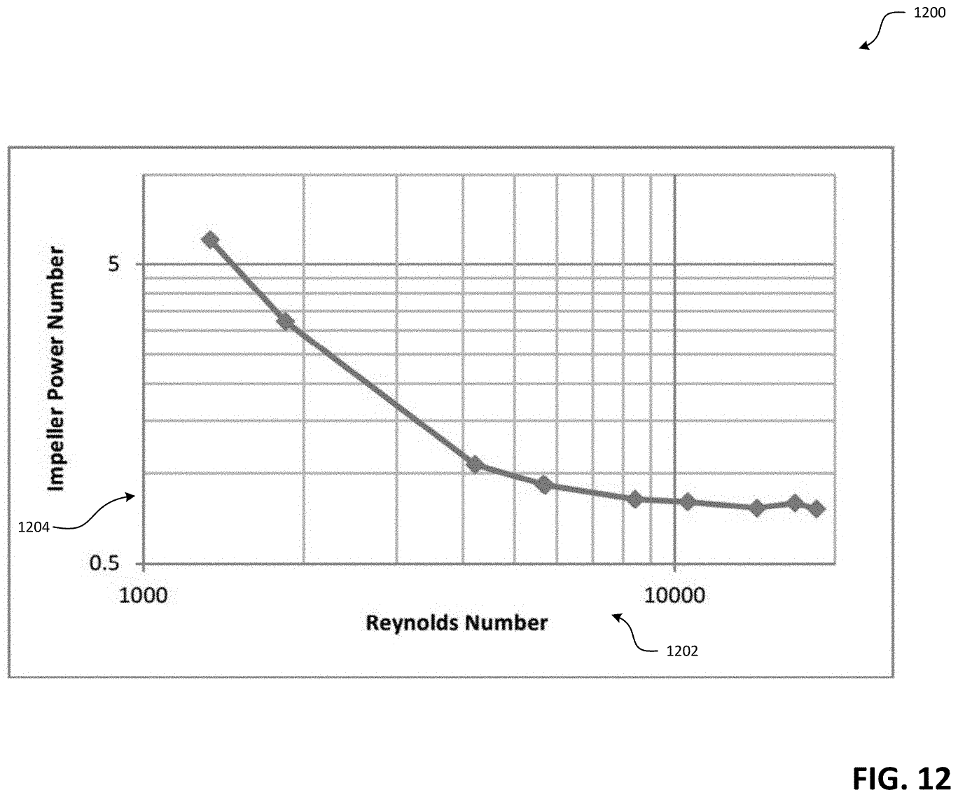

FIG. 12 is a graph 1200 including an X-axis 1202 representing Reynolds numbers and a Y-axis 1204 representing impeller power. Specifically, FIG. 12 shows impeller power number vs. Reynolds Number for the second bioreactor 106 having a volume of approximately 0.5 L and a working volume of approximately 300-ml with DI water at approximately 26°. As shown in FIG. 12, as the Reynolds Number increases, the impeller power number decreases.

FIG. 12 shows the same results as included in FIG. 11, plotted as impeller power number versus Reynolds number. The correlation has similarities to those found for anchored helical ribbons, double helix, and other impellers that have very high impeller-diameter-to-tank-diameter (Di/T) ratios. For such impellers, the laminar regime, wherein power number is inversely proportional to Reynolds number, is often observed for Reynolds numbers up to roughly 100. This is in contrast to typical turbines and propellers, with typical Di/T ratios of 0.25-0.5, wherein the laminar regime is observed for Reynolds numbers below 10. Using the second bioreactor 106 having a volume of approximately 0.5 L, with a Di/T ratio of 0.85 and an unusual vertical wheel, the laminar regime appears to persist up to a Reynolds number of approximately 4000. At Reynolds numbers above 10,000, the fully turbulent regime is reached, with a constant power number averaging 0.78. Unlike in unbaffled vessels with flat-paddles or other impellers, the power number does not continue to slowly decline beyond a Reynolds number of approximately 10,000. This may be due to the lack of vortexing with the vertical impeller configuration. Between the second and third points in FIG. 12, at Reynolds numbers of 1850 and 4214, respectively, the slope of a power-law fit is −1.3. The fact that the slope through the first three points is steeper than negative 1.0, as would otherwise be expected for the laminar regime, is most likely due to experimental challenges regarding power measurements at these very low levels. This slope, as well as the persistence in the laminar regime at moderately high Reynolds numbers, is reproducible under the protocols presented here.

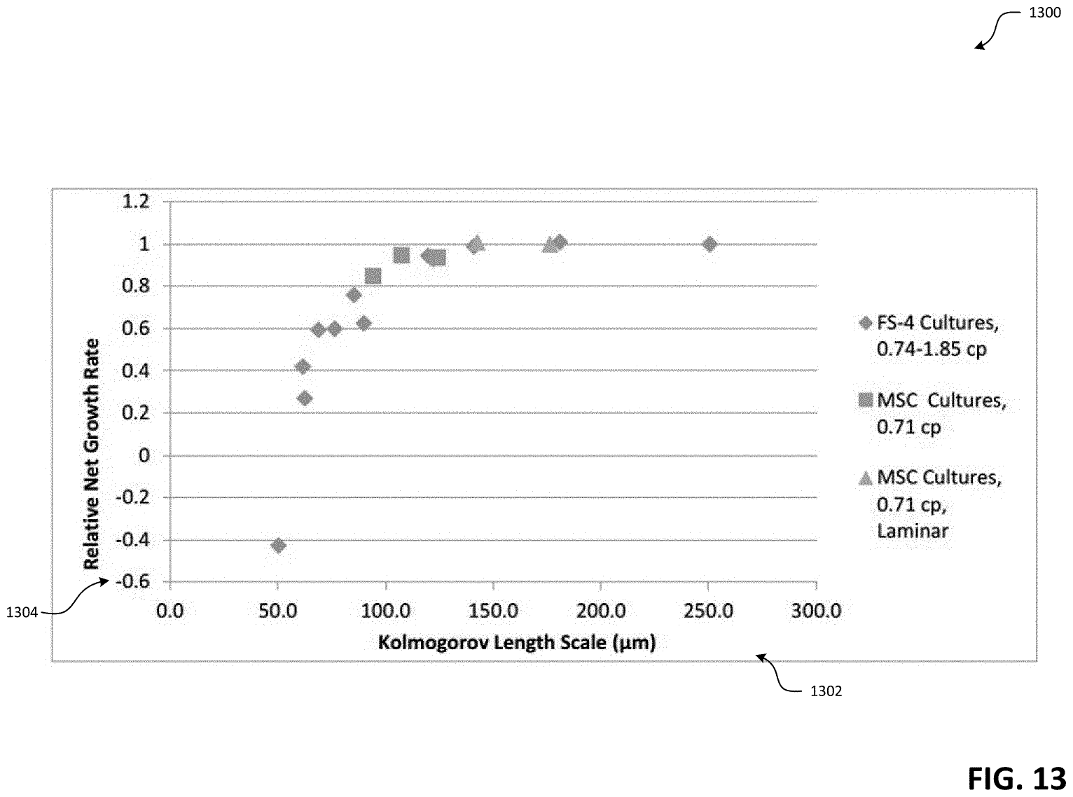

FIG. 13 is a graph 1300 including an X-axis 1302 representing Kolmogorov Length Scale (μm) and a Y-axis 1304 representing the relative net growth rate. Specifically, the graph 1300 illustrates effect of Kolmogorov eddy length on relative growth extent for FS-4 cells growing on cytodex 1 microcarriers when stirred spinner STRs at various viscosities, as well as MSCs on Solohill plastic plus microcarriers in the second bioreactor 106 having a volume of approximately 0.5 L (including laminar flow regime). As shown in FIG. 13, as the Kolmogorov Length Scale number increases, the relative net growth rate also increases and then becomes relatively consistent after the relative net growth rate is approximately 1.0.

To obtain the data plotted in the graph 1300 of FIG. 13, the correlation previously shown in FIG. 12 was used and data published on microcarrier cultures in vertical wheel bioreactors was analyzed in terms of power input via Kolmogorov length scale. The results are shown in FIG. 13 and are in line with previously published results. When cells are grown on microcarriers having a diameter of between approximately 150 and approximately 200-micron in either horizontal impeller or vertical-wheel impeller bioreactors, hydrodynamic damage becomes apparent when the Kolmogorov length scale (based upon average power/mass) hits 130 microns or less. At a kinematic viscosity of 0.0071 cm²/sec, this translates to an average power per mass of 12.5 cm²/sec³.

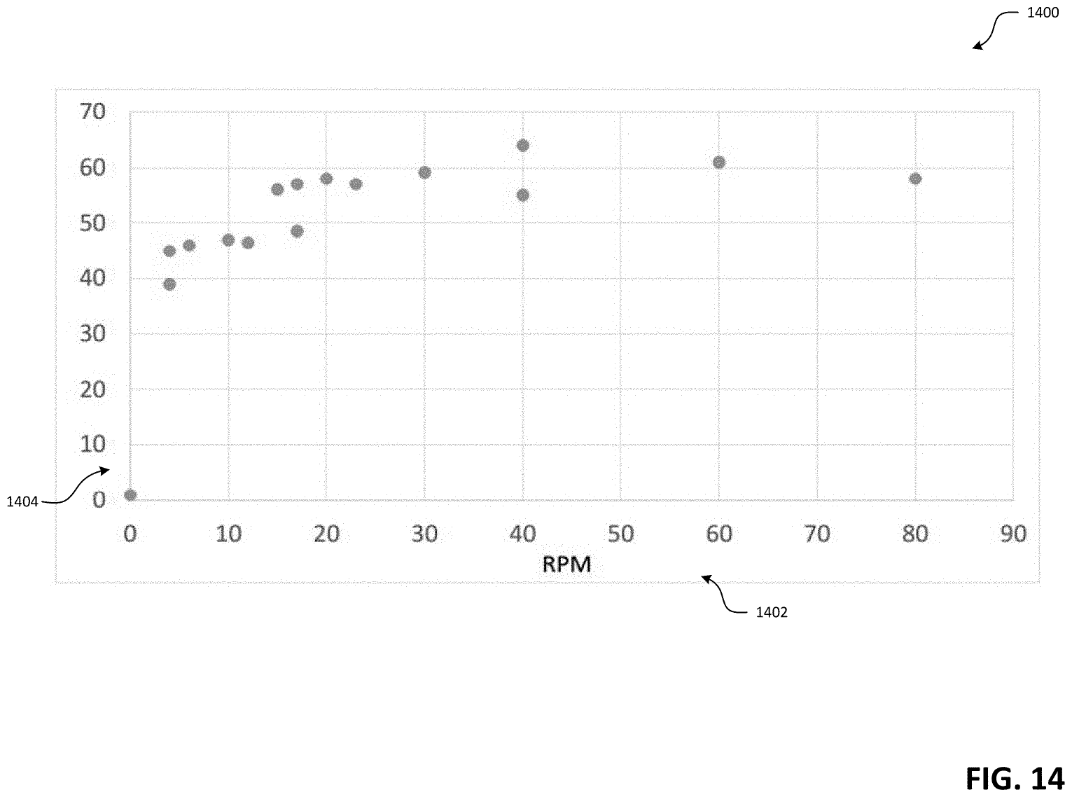

The correlation shown in FIG. 13 was also used to determine the power used for microcarrier suspension across a series of different scale vertical-wheel bioreactors such as, for example, the second bioreactor 106. FIG. 14 is a graph 1400 presenting sets of results from microcarrier suspension studies where the X-axis 1402 represents revolutions per minute (RPMs). To determine the power used for the different bioreactors, microcarriers were suspended at low concentration in phosphate-buffered-saline in the bioreactors 102, 106. At various agitation levels, small samples were withdrawn from near the surface of each bioreactor 102, 106, and the number of microcarriers per ml of sample was counted via an inverted microscope. The agitation using the mixer 116 and/or the wheel 120 was increased until a clear plateau in counts was obtained. FIG. 14 represents the results obtained using the second bioreactor 106 having a volume of approximately 0.1 L. The counts plateau with agitation of 15-20 RPMs or higher, indicating that the microcarriers are in suspension and/or that all or at least some of the microcarriers are fully suspended at any or at least some agitation rates above, within and/or at the range of 15-20 RPMs. This was also confirmed visually.

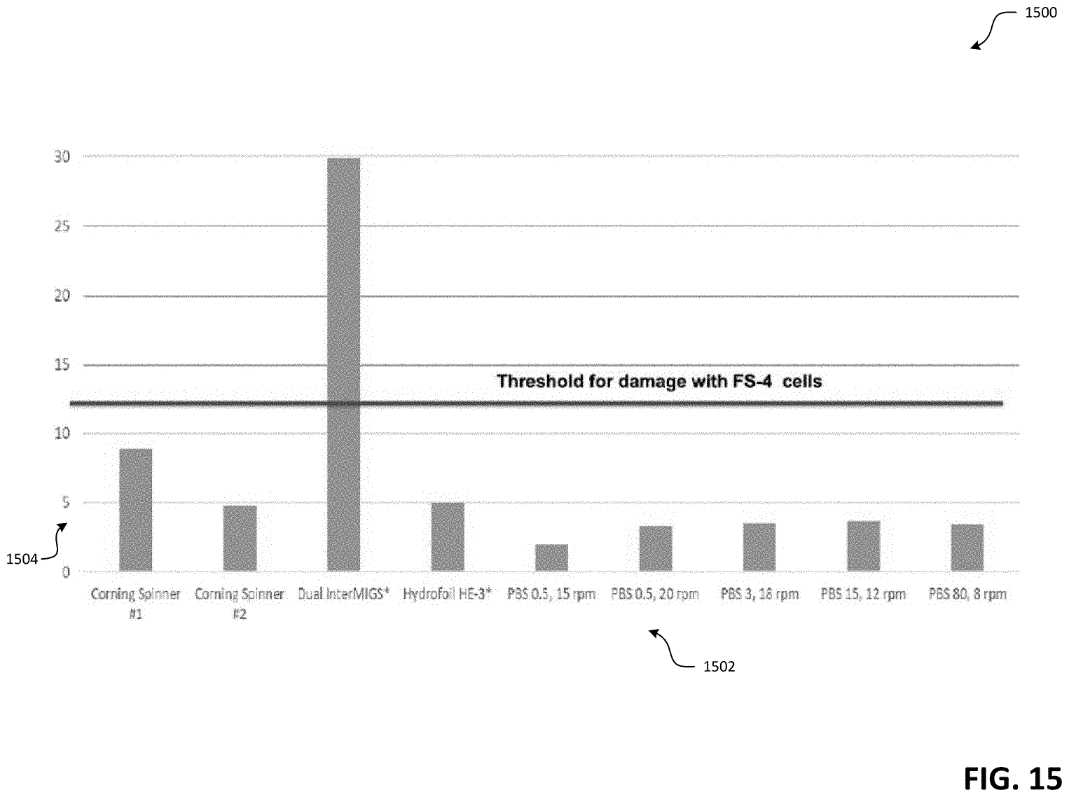

FIG. 15 is a graph 1500 including an X-axis 1502 mentioning different bioreactors and the associated agitation rates (rpms) and a Y-axis 1504 representing the average power per mass levels required for microcarriers suspension in various bioreactors. Translating the minimum rotations per minute (RPMs) used for microcarrier suspension to power levels using correlations such as shown in FIG. 14, one can calculate the average power per mass used to suspend microcarriers in various bioreactors. Vertical-wheel bioreactors across different scales can suspend microcarriers at very low levels of power per mass, in the range of between approximately 2 cm²/sec³ and approximately 3.5 cm²/sec³, which is far below the damage threshold of 12.5 cm²/sec³ for FS-4 cells and many other cell lines, including MSCs, than a higher quality stirred-tank reactors (STRs) that use horizontal-impeller mixing. Data shown for vertical-wheel bioreactors of various sizes along with Corning spinner STRs and a range of 20-L STRs. As mentioned previously, at 12.5 cm²/sec³, the eddies approach approximately 130 microns in size, which is sufficiently small enough (approximately two-thirds the diameter of the microcarrier or less) to have a shearing effect on surface-attached cells.

As previously explained, cell aggregates of various cell types have similar diameter and density to plastic microcarriers, such as the Solohill microcarriers used in the suspension studies shown in FIGS. 13-15. Therefore, it may be inferred that the power per mass range of between approximately 2 cm²/sec³ and approximately 3.5 cm²/sec³ is the minimum baseline capable of also suspending cell aggregates across the same range of vertical-wheel bioreactors, from 0.1 L to 80 L. However, other power per mass ranges may be used. However, other power per mass ranges may prove suitable.

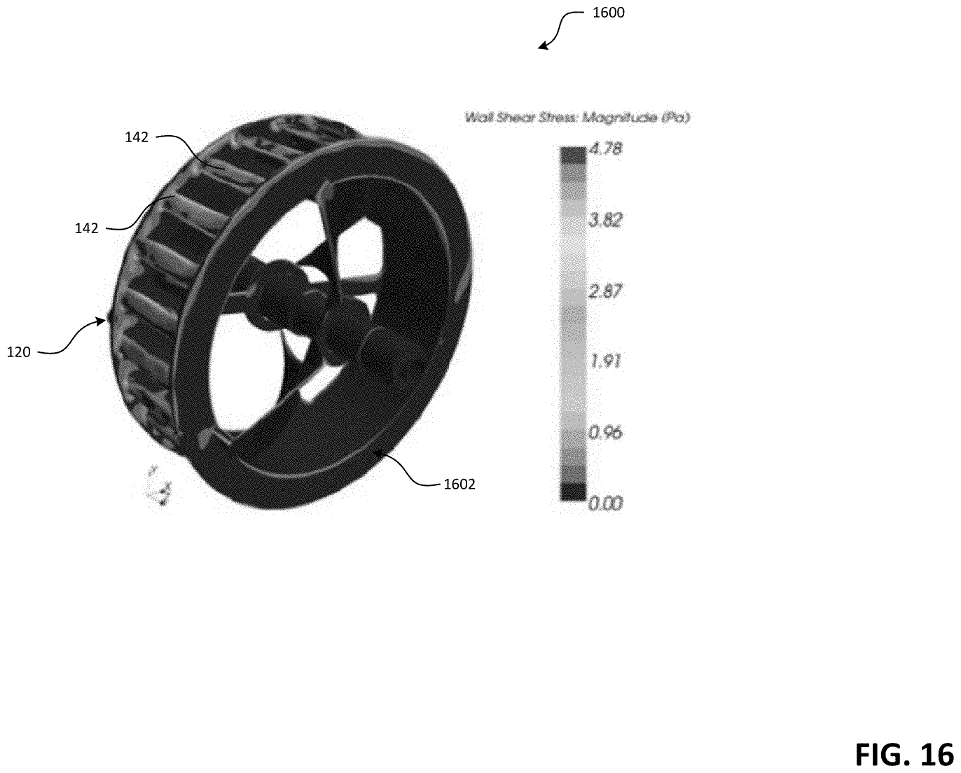

FIG. 16 shows results 1600 of a computational fluid dynamics (CFD) analysis performed on the first bioreactor 102 having a volume of approximately 3 L. The results 1600 of FIG. 16 show that there are relatively consistent and low levels of hydro dynamic shear stress on the surface of the wheel 120 (pneumatically driven impeller) during liquid mixing at 3 L scale. As shown, sides 1602 of the wheel 120 experience approximately 0.0 Pascal (Pa) of sheer stress while the radial flow blades 142 of the wheel 120 experience between approximately 0.0 Pascal (Pa) of sheer stress and approximately 2.0 Pascal of sheer stress.

Additionally, tests were performed at various scales of vertical-wheel bioreactors and the results show low hydrodynamic shear stress levels on the surface of the wheel 120. The analysis that provided the results 1600 of FIG. 16 were performed on the vertical-wheel bioreactor 102, 106 using an impeller version that is pneumatically rotated through buoyancy of streaming air bubbles as opposed to magnetic coupling. However, because the wheel 120 and U-shaped vessel dimensions, aspect ratios, and bioreactor functions are similar or the same between the first bioreactor 102 and the second bioreactor 106, the shear stress levels on the surface of the impeller would be relatively low regardless of magnetic or pneumatic mixing.

The power per mass range of between approximately 2 cm²/sec³ and approximately 3.5 cm²/sec³ is sufficient to suspend cell aggregates and also translates to minimum agitation rates that can create a homogeneous EDR within the vertical-wheel bioreactor. Homogeneous EDR is the prerequisite to a uniform mixing environment, which will promote the formation of uniformly shaped cell aggregates. The size and shape of PSC aggregates have a direct effect on the efficiency of cell expansion and subsequent directed differentiation. If an aggregate becomes too large or misshapen, nutrients or differentiation factors may be unable to diffuse into its center, leading to unwanted cell death or heterogeneous differentiation. A homogeneous mixing environment is conducive to the formation of spherical cell aggregates of equal size. Achieving a narrow range of diameters and uniform spherical shapes for cell aggregates will increase the productivity of both PSCs expansion and differentiation as well as the yield and quality of the target cells as final products.

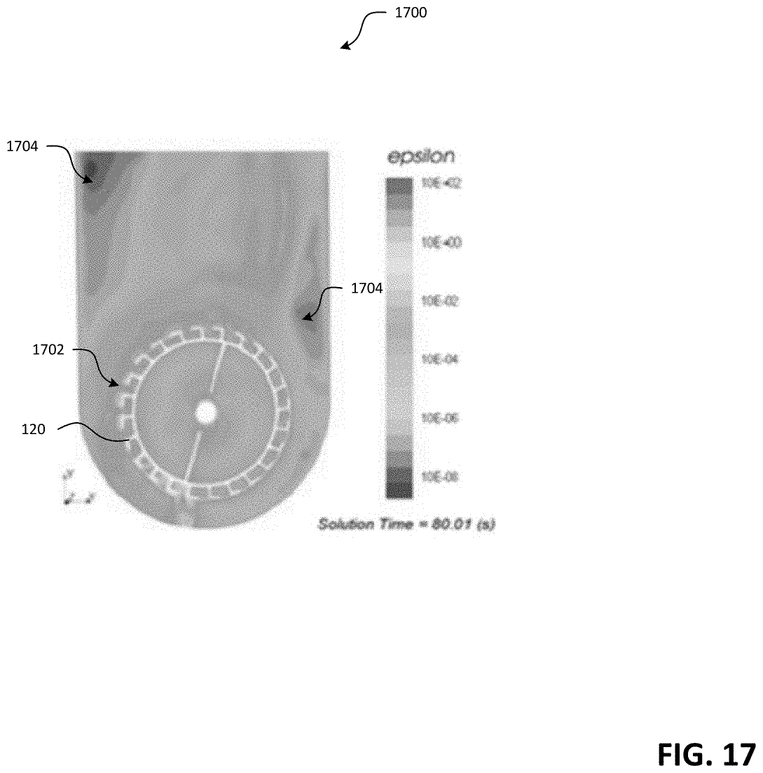

FIG. 17 shows additional results 1700 obtained from computational fluid dynamics (CFD) analysis using the first bioreactor 102 and/or the second bioreactor 106 of FIG. 4 and show that substantially homogeneous distribution of turbulent energy dissipation rates can be obtained for the vertical-Wheel mixing (pneumatically driven impeller 120) in the U-shaped containment vessel 104, 108. The results show that the range of turbulent energy dissipation rates (EDR) is relatively narrow and toward the middle of the spectrum (between approximately 10E-02 and approximately 10E-06), that the dissipation rates are uniformly distributed throughout the containment vessel 104, 108 without zones that are drastically different, and that the rates are shown in units of epsilon (m² s⁻³).

The results 1700 of FIG. 17 also indicate how vertical-wheel mixing, in conjunction with a U-shaped vessel such as provided by the first bioreactor 102 and/or the second bioreactor 106, results in a homogeneous mixing environment with a narrow range of EDR inside the containment vessel 104, 108 (as indicated by the area 1702 surrounding the wheel 120, with some areas 1704 have an EDR rate of approximately 10E-08, in FIG. 17). The results 1700 and the associated model were obtained using the pneumatically-driven version of vertical-wheel bioreactor 102, 106, but the same or similar considerations may apply. For example, there would be similarities in homogeneity for the CFD models between pneumatic versus magnetic drive mixing.

Pneumatically-driven impeller mixing showed homogeneous and EDR that could be scaled from approximately 0.1 L to approximately 500 L working volumes and it can be predicted that magnetically-driven impeller mixing, in the same U-shape containment vessel 104, 108, can achieve similar or the same homogeneity and scalability.

The computational fluid dynamics (CFD) analysis using the first bioreactor 102 and/or the second bioreactor 106 of fluid mixing based on the combination of the vertical-wheel impeller 120 and the U-shaped containment vessel 104, 108 indicates a narrow range of homogeneous turbulent energy dissipation rates throughout the containment vessel 104, 108, as well as consistent hydrodynamic shear stress on the surface of the impeller 120, creating the threshold uniform mixing environment for PSC aggregates. This was confirmed by observing uniform size and shape distribution of PSC aggregates grown in small-scale vertical-wheel bioreactors.

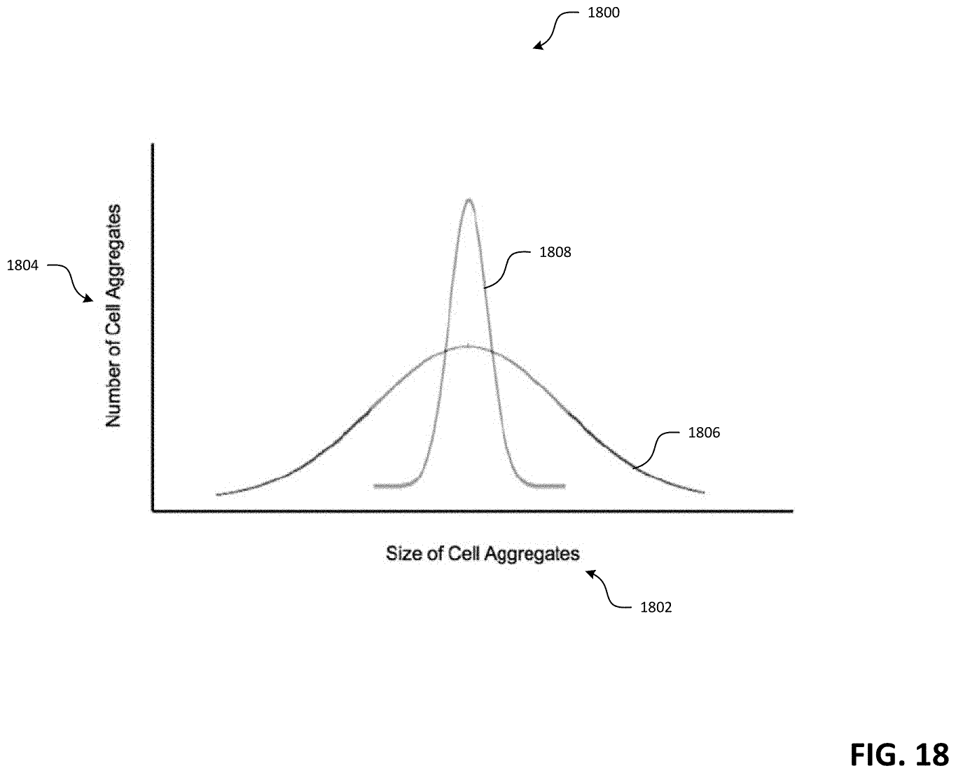

FIG. 18 is a graph 1800 including an X-axis 1802 representing the size of cell aggregates and a Y-axis 1804 representing the number of cell aggregates. The graph 1800 includes a first curve 1806 associated with a bioreactor having a wide range gradient of dissipation of energy rates and cell aggregates produced having inconsistent sizes and/or shapes and a second curve 1808 associated with a bioreactor having a homogeneous distribution of dissipation energy rates and cell aggregates having similar sizes and/or shapes. The graph 1800 of FIG. 18 indicates that variation of PSC aggregate sizes grown in a bioreactor with homogeneous turbulent EDR would be much narrower, represented by the steep bell curve, than the variation of PSC aggregate sizes grown in a bioreactor with a wide gradient of turbulent EDR represented by the gradual bell curve.

The size and shape of PSC aggregates are significantly affected by the fluid mixing environment inside the bioreactor during a cell culture process. In particular, there is an inverse correlation between EDR and average diameter of resulting cell aggregates: high EDR results in smaller average diameters of cell aggregates, while low EDR results in larger diameters. In order to achieve spherical PSC aggregates of consistent diameter, a narrow range of turbulent EDR is used. A bioreactor such as the first and/or second bioreactors 102, 106 with mixing mechanism (the mixer 116) that results in a broad range of turbulent EDR throughout the bioreactor containment vessel 104, 108 will produce a wide variation of cell aggregate sizes, which can negatively impact efficiency of cell expansion and differentiation.

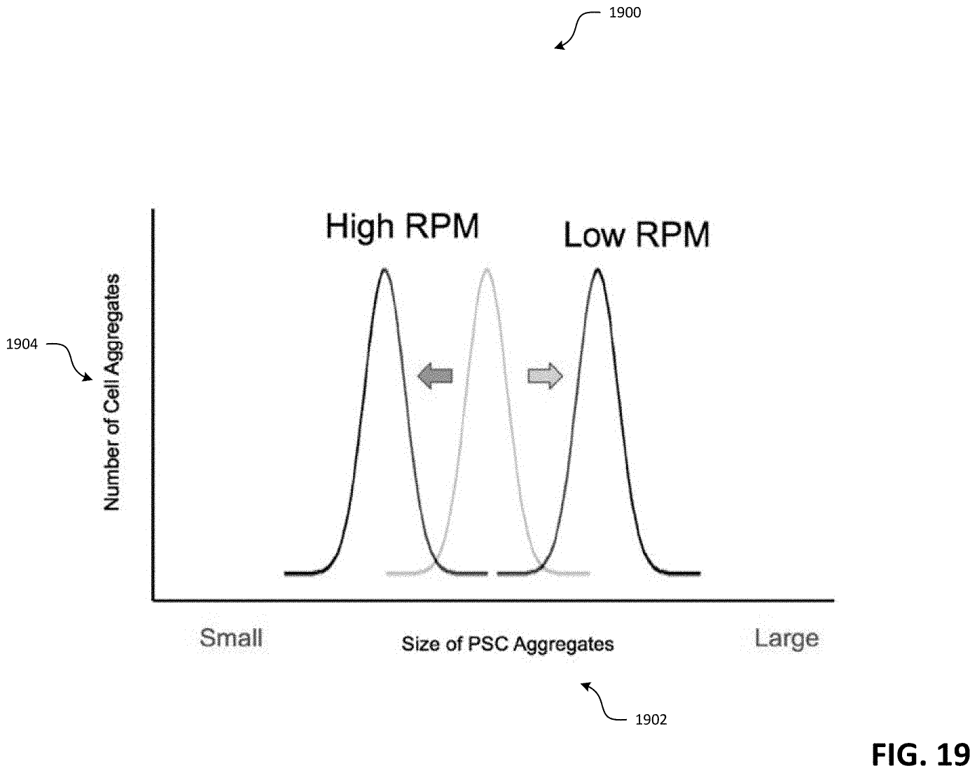

FIG. 19 is a graph 1900 including an X-axis 1902 representing the size of PSC aggregates and a Y-axis 1904 representing the number of cell aggregates. Specifically, the graph 1900 illustrates the controllability of PSC aggregate sizes in a bioreactor by varying agitation rates. As shown, by increasing the RPM rates, the size of the PSC aggregates decreases and, by decreasing the RPM rate, the size of the PSC aggregates increases. The average diameter of PSC aggregates can be controlled by adjusting agitation rate in a bioreactor with homogeneous EDR. This is desirable as different types of PSCs can have different threshold aggregate diameters necessary to increase efficiency of cell expansion or differentiation.

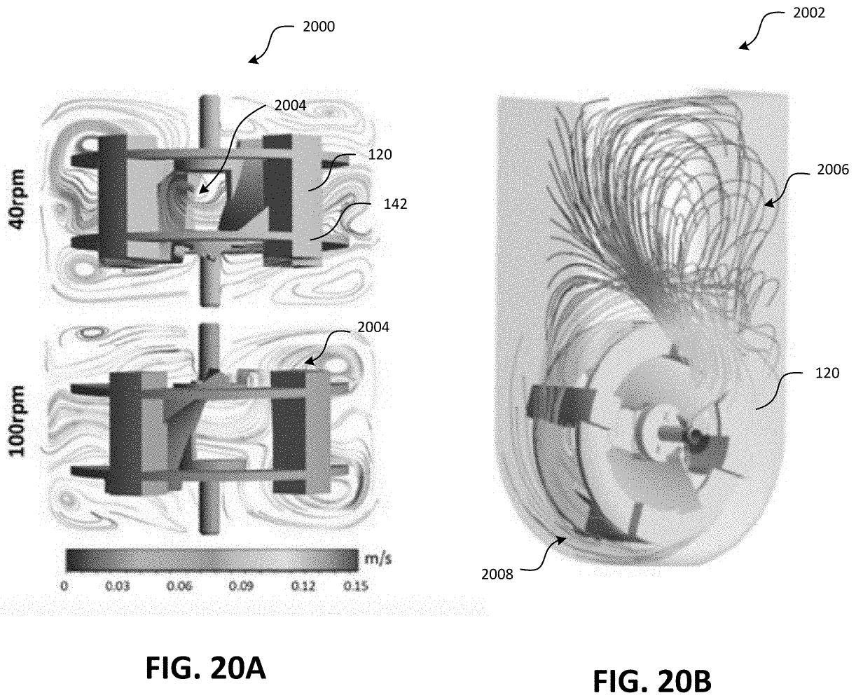

FIG. 20A shows a top view of computational fluid dynamics (CFD) analysis results 2000 for a Lemniscate liquid flow pattern and velocity stream lines for the second bioreactor 106 including the vertical impeller wheel 120 having a volume of approximately 0.1 L, with some of the tests being performed with the wheel 120 rotating at approximately 40 rpms and some of the tests being performed with the wheel 120 rotating at approximately 100 rpms.

FIG. 20B shows a side isometric view of computational fluid dynamics (CFD) analysis results 2002 for a Lemniscate liquid flow pattern and velocity stream lines for the second bioreactor 106 including the vertical impeller wheel 120 having a volume of approximately 0.1 L with the tests being performed with the wheel 120 rotating at approximately 60 rpms.

Referring to both FIGS. 20A and 20B, the results 2000, 2002 show a pattern of liquid flow throughout the entire volume of the U-shaped containment vessel 108, which is in a lemniscate or “figure-8” pattern. This is a unique streamline flow-pattern compared to typical funnel or “tornado” pattern of liquid flow in STRs and may occur due to the combination of the vertical-wheel impeller 120 and the U-shaped containment vessel 108, and may enable the uniform and scalable mixing environment with homogeneous energy dissipation rates and consistently low sheer stress levels. As shown in FIG. 20A, turbulent eddies 2004 flow through the wheel 120 and throughout the containment vessel 104, 108. In some implementations, all or substantially all of the aggregates travel throughout the containment vessel 104, 108 and experience the same or at least similar hydrodynamic conditions (e.g., EDR and sheer stress). As such, the aggregates have substantially equal or similar sizes and/or shapes. As shown in FIG. 20A, the eddies 2004 of the second bioreactor 106 operated at 40 RPMs are moving at between approximately 0.03 m/s and approximately 0.06 m/s with the velocity of the eddies 2004 closer to the blades 142 moving at approximately 0.06. As shown in FIG. 20A, the eddies 2004 of the second bioreactor 106 operated at 100 RPMs are moving at between approximately 0.03 m/s and approximately 0.15 m/s with the velocity of the eddies 2004 closer to the blades 142 moving at between approximately 0.012 m/s and approximately 0.015 m/s. As shown in FIG. 20B, eddies 2006 farther away from the wheel 120 are moving at between approximately 0.0 m/s and approximately 0.06 m/s, while eddies 2008 closer to the wheel 120 are moving between approximately 0.06 m/s and approximately 0.11 m/s.

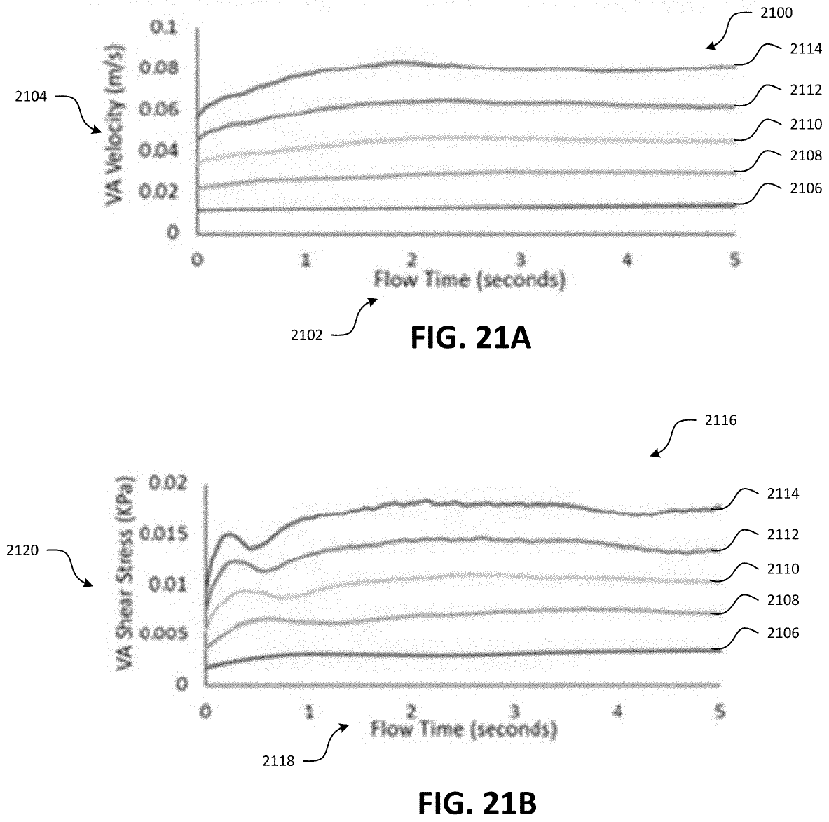

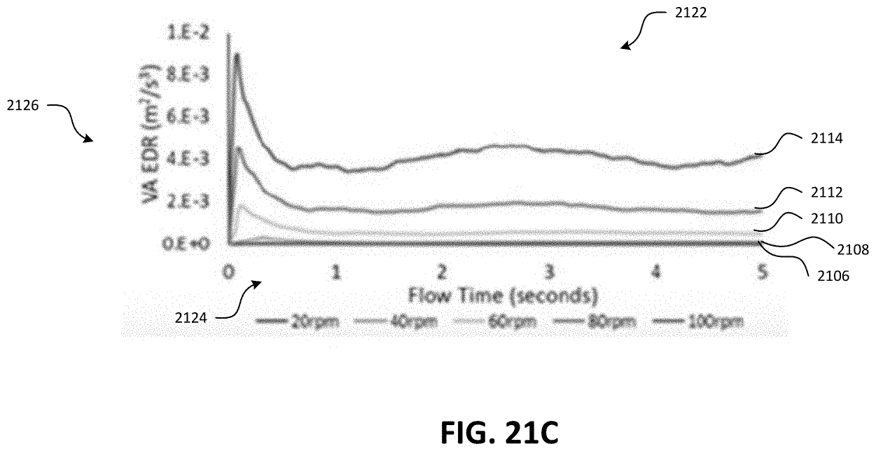

FIG. 21A is a graph 2100 including an X-axis 2102 representing flow time in seconds and a Y-axis 2104 representing velocity. The graph 2100 includes a first line 2106 associated with the wheel 120 operating at 20 rpms, a second line 2108 associated with the wheel 120 operating at 40 rpms, a third line 2110 associated with the wheel 120 operating at 60 rpms, a fourth line 2112 associated with the wheel 120 operating at 80 rpms, and a fifth line 2114 associated with the wheel 120 operating at 100 rpms. As shown, the velocity value is relatively consistent after approximately one second of flow time.

FIG. 21B is a graph 2116 including an X-axis 2118 representing flow time in seconds and a Y-axis 2120 representing sheer stress and FIG. 21C is a graph 2122 including an X-axis 2124 representing flow time in seconds and a Y-axis 2126 representing EDR. As shown in FIG. 21B, the shear stress value is relatively consistent after approximately one and a half seconds of flow time. As shown in FIG. 21C, the EDR value is relatively consistent after approximately two seconds of flow time.

Referring to FIGS. 21A, 21B, and 21C, these graphs 2100, 2116, 2122 show how quickly fluid stream line velocity, shear stress, and EDR reach their threshold (e.g., maximum) steady state values once the impeller 120 begins initial rotation from a static position or a stopped position. Steady state for all three fluid dynamic properties was reached in approximately three seconds, regardless of RPM used. This is useful to quickly resuspend any cell aggregates that may have settled during a cell culture process step such as medium exchange. The second bioreactor 106 having a volume of approximately 0.1 L was used when performing the tests to obtain the data displayed in the graphs 2100, 2116, 2122.

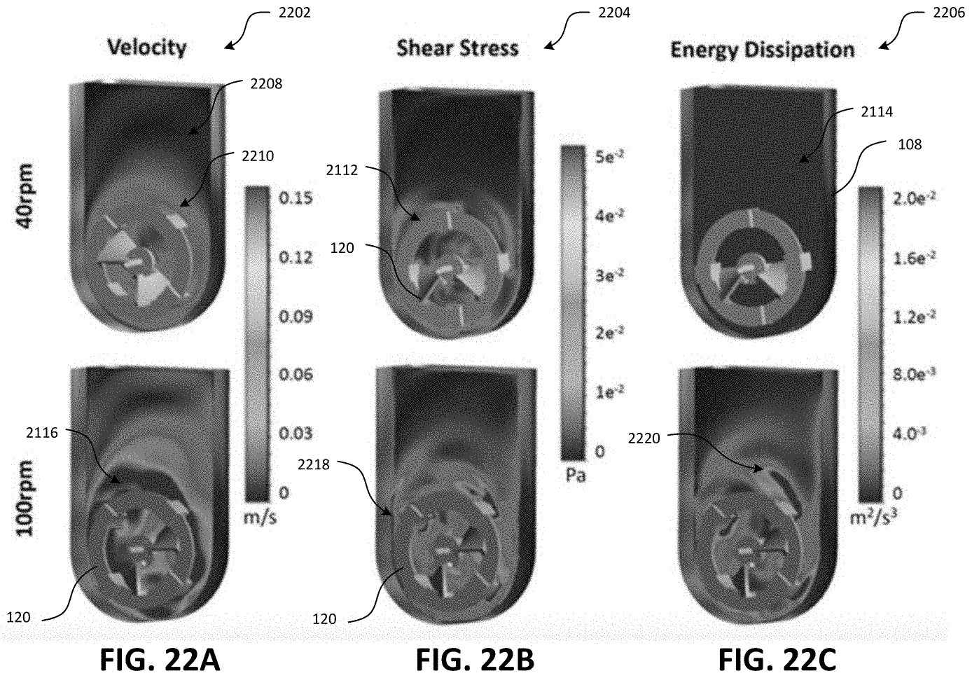

FIG. 22A show computational fluid dynamics (CFD) analysis results 2202 related to velocity with the wheel 120 rotating at approximately 40 rpm and at approximately 100 rpm, FIG. 22B show computational fluid dynamics (CFD) analysis results 2204 related to sheer stress with the wheel 120 rotating at approximately 40 RPMs and at approximately 100 RPMs, and FIG. 22C show computational fluid dynamics (CFD) analysis results 2206 related to energy dissipation with the wheel 120 rotating at approximately 40 RPMs and at approximately 100 RPMs. Thus, FIGS. 22A, 22B, and 22C show the relationship of velocity, shear stress, and EDR at 40 and 100 RPMs with tests being performed using the second bioreactor 106 having a volume of approximately 0.1 L.

With reference to FIGS. 22A, 22B, and 22C, at both 40 RPMs and 100 RPMs (and it can be inferred for all RPMs, substantially all RPMs, and/or some RPMs), the variation of shear stress and EDR do not increase at the same rate as velocity (when power input to impeller is increased). In fact, at 40 RPM, the EDR is almost completely homogeneous (see areas 2208 and 2210). At reference number 2208, the velocity is between approximately 0.0 m/s and approximately 0.03 m/s and at reference number 2210, the velocity is between approximately 0.03 m/s and approximately 0.09 m/s. At reference number 2112 of FIG. 22B, the sheer stress around the wheel 120 being operated at approximately 40 RPMs is between approximately 1 E-2 and approximately 3E-2 and at reference number 2114 of FIG. 22C, the energy dissipation value is approximately 0.0 m²/m³ and is substantially consistent throughout the containment vessel 108.

At 100 RPM and as shown at reference number 2116 of FIG. 22A, the velocity around the wheel 120 is between approximately 0.09 m/s and approximately 0.15 m/s. As shown in FIG. 22B, there is much greater variation in velocity when the wheel 120 is operated at 100 RPMs versus as compared to when the wheel 120 is operated at 40 RPMs but there are minimal zones of relatively high shear stress or EDR (see area 2218 where the sheer stress is between approximately 3E-2 Pa and approximately 4E-2 Pa). Therefore, increasing power input to the impeller wheel 120 affects velocity more so, but a substantially uniform mixing environment is maintained even at higher RPMs. It can also be predicted that a similar relationship/behavior between these three fluid conditions, velocity, sheer stress, and energy dissipation will be substantially consistent during scale up to larger bioreactor volumes such as, for example, the first bioreactor 102. Moreover, as shown in FIG. 22C when the second bioreactor 106 is operated at approximately 100 RPM, the energy dissipation value is between approximately 4.0E-3 m²/s³ and approximately 2.0E-2 m²/s³.

There may be a large difference between the maximum and minimum values for velocity, shear stress, and EDR (especially velocity) as power input increases. The maximum and minimum values do not actually provide much input to the bioreactor environment because a small fraction of the bioreactor ever experiences these conditions. What typically affects the bioreactor environment is the average value, and whether a high majority percent of the bioreactor 102, 106 is operating at a reasonable average value that will not negatively impact cell aggregate formation.

The effect of agitation rates on PSC aggregate diameters and corresponding superior biological performance, has been demonstrated at small scale (0.1 L) vertical-wheel bioreactors such as the second bioreactor 106 and compared to STRs. The uniform mixing environment created by a vertical-wheel impeller 120 is in stark contrast to the non-homogeneous environment created by at least some horizontal impeller mixing in stirred-type bioreactors (STRs).

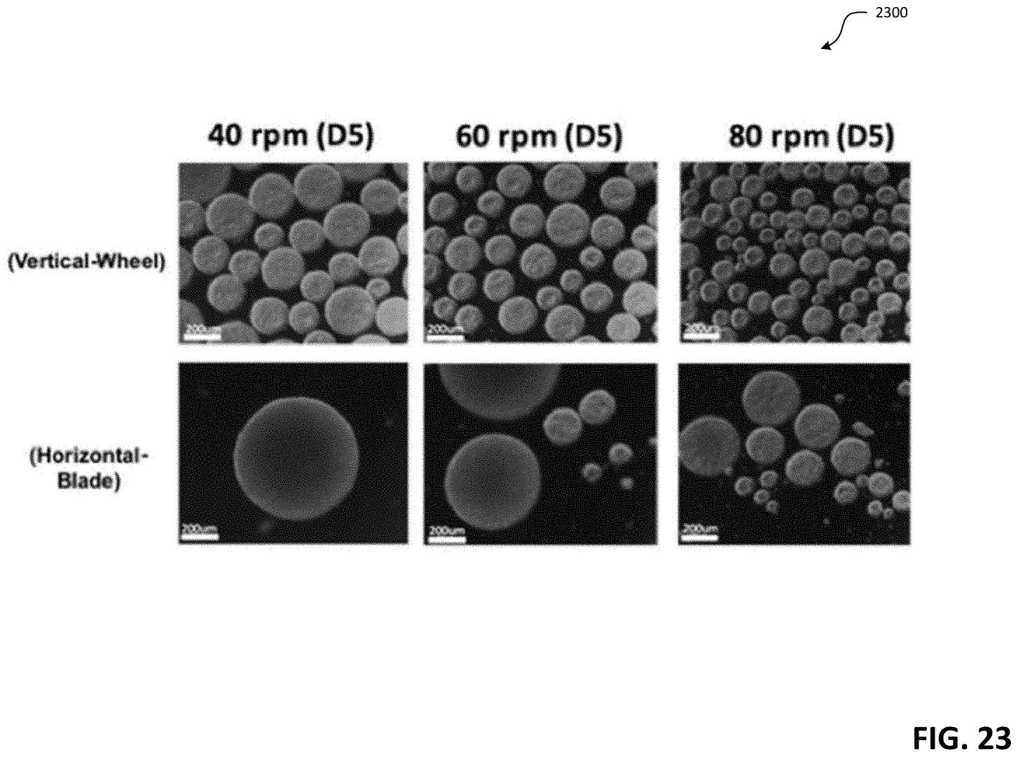

FIG. 23 illustrates results 2300 obtained when growing cells in a bioreactor including a vertical wheel, such as the bioreactors 102, 106 of FIG. 4 and a bioreactor including a horizontal blade at different agitations rates, 40 RPMs, 60 RPMs, and 80 RPM. More specifically, the results 2300 allow for a comparison of iPSC aggregate diameters and morphology with different agitation rates in the second bioreactor 106 having a volume of approximately 0.1 L and a bioreactor having a horizontal blade.

In examples where iPSCs were seeded as single cells and expanded for five days, vertical-wheel mixing such as that provided by the bioreactors 102, 106 disclosed was shown to result in narrower ranges of aggregate diameters and much more uniform aggregates compared to when the horizontal-blade was used. This inverse correlation between the RPMs of the impeller wheel 120 and the cell aggregate diameter was also confirmed using the second bioreactor 106 having a volume of approximately 0.1 L.

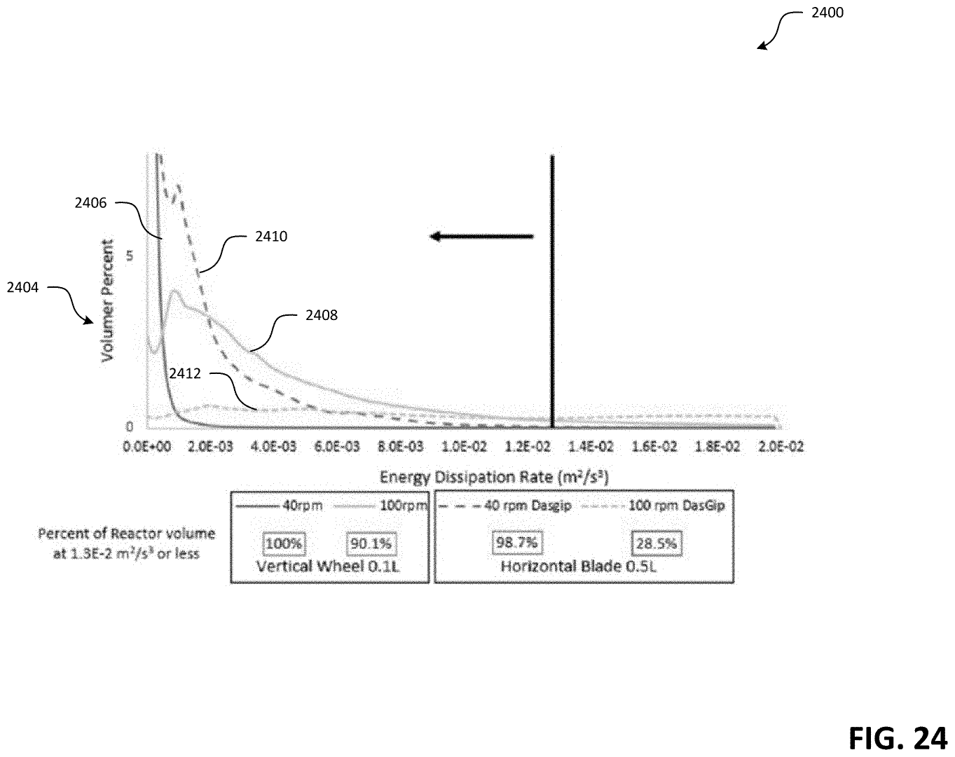

FIG. 24 is a graph 2400 including an X-axis 2402 representing energy dissipation rates (EDRs) and a Y-axis 2404 representing volume percent. More specifically, FIG. 24 shows the difference in volume percentages under this EDR threshold reactor for 0.1 L vertical-wheel bioreactor and horizontal blade spinner, at 40 and 100 rpms.

The graph 2400 includes a first line 2406 associated with operating the second bioreactor 106 having a volume of approximately 0.1 L at approximately 40 RPMs, a second line 2408 associated with operating the second bioreactor 106 having a volume of approximately 0.1 L at approximately 100 RPMs, a third line 2410 associated with operating the horizontal wheel bioreactor having a volume of approximately 0.5 L at approximately 40 RPMs, a fourth line 2412 associated with operating the horizontal wheel bioreactor having a volume of approximately 0.5 L at approximately 100 RPMs. The first line 2406 has a relatively smooth and sharply sloped distribution without significant bumps (e.g., outliers) and a majority of the EDR values occur before 2.0E-3 and, thus, the EDR values are relatively similar and the cells grown may have similar shapes and/or sizes. The second, third, and fourth lines 2408, 2410, 2412 have more shallow lines and, thus, a broader range of EDR values.

The results show that successful PSC aggregate growth and consistency in 0.1 L bioreactor has been measured to occur when at least 90% of the working volume maintains an energy dissipation rate of 1.30E-2 m²/s³ or less.

Still referring to FIG. 24, at 40 rpm, approximately 100% of the 0.1 L vertical-wheel bioreactor volume is under 1.30E-2 m²/s³, and the 0.5 L horizontal-blade spinner has similar 99% under that EDR. However, there is significant difference at 100 rpm: 90% of vertical-wheel bioreactor volume is still under 1.30E-2 m²/s³, but the horizontal-blade spinner has about 28.5% under the 1.30E-2 m²/s³ EDR value. This means that the horizontal-blade spinner has significantly heterogeneous EDR at higher RPMs, which will lead to non-uniform size and shape of PSC aggregates. In one example, the vertical-wheel mixing in a U-shaped vessel has the fluid dynamics that allow for wide range of RPMs while still maintaining homogeneous EDR of a preferred 90% volume. Put another way, the horizontal blade has a relatively “broad” distribution with “multiple peaks” which leads to a heterogeneous distribution of aggregate sizes and shapes (see, FIG. 23).

40 RPMs corresponds to slightly more than the power per mass range of between approximately 2 cm²/sec³ and approximately 3.5 cm²/sec³ that may be the minimum requirement to fully suspend microcarriers and cell aggregates. While 100 RPMs is on the upper end of what would typically be used for a cell culture process, it still achieves a mixing environment with much more homogeneous EDR and consistently lower shear stress compared to what horizontal-impeller mixing achieves at same agitation rate.

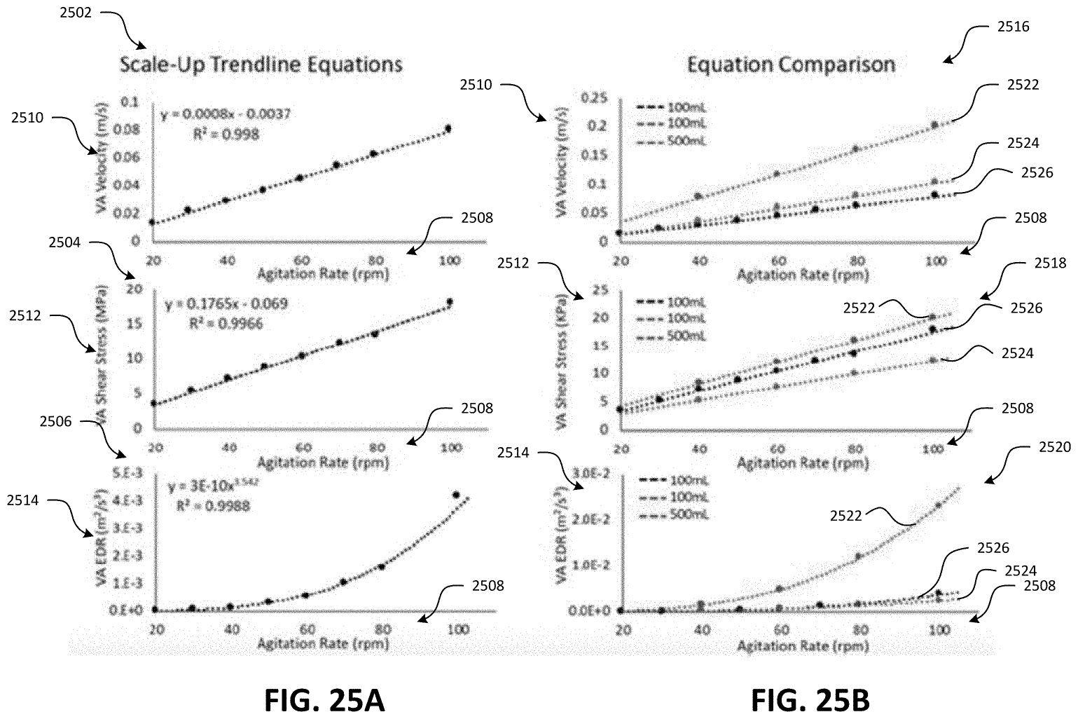

FIG. 25A shows graphs 2502, 2504, 2506 of scale-up trendline equations when using the second bioreactor 106 having a volume of approximately 0.1 L. Each of the graphs 2502, 2504, 2506 has an X-axis 2508 representing the agitation rate (RPMs) and the graph 2502 has a Y-axis 2510 representing velocity, the graph 2504 has a Y-axis 2512 representing shear stress, and the graph 2506 has a Y-axis 2514 representing EDR. As shown in each of the graphs 2502, 2504, 2506, the velocity, shear stress, EDR values increase as the agitation rate increases.

FIG. 25B shows graphs 2516, 2518, 2520 including a first line 2522 associated with results obtained using the second bioreactor 102, a second line 2524 associated with results obtained using a NDS bioreactor having a horizontal-blade spinner, and a third line 2526 associated with results obtained using a DasGip® bioreactor having a horizontal-blade spinner.

As shown in FIGS. 25A and 25B, shear stress is confirmed to increase linearly with agitation rate (velocity) while EDR increases exponentially with velocity. When compared to two kinds of horizontal-blade spinners (compare line 2522 to lines 2524 and 2526), the vertical-wheel bioreactor 106 has similar or lower average velocity, shear stress, and EDR across wide range of agitation rates at the 0.1 L scale. However, the superiority of uniform mixing environment of vertical-wheel bioreactor 106, and subsequent uniform cell aggregate formation, becomes much more pronounced as volume increases in scale.

Using the volume average values for EDR, it is possible to define operating agitation rates for particular cell culture. For example, if one wanted to define operation between approximately 40 rpm and approximately 80 rpm at the 0.1 L scale, one would operate with a volume average EDR between approximately 5.67E-5 m²/s³ and approximately 1.59E-3 m²/s³. By performing small-scale experiments in the vertical-wheel bioreactors 106, the EDR range that produces desired aggregates of threshold diameter for a given PSC type can be determined, and then the agitation rate to be used to recreate that EDR range at larger scale can be calculated using, for example, the controller 124. This will enable that the PSC aggregates experience a similar or the same mixing environment in any size vertical-wheel bioreactor, which is important for a scalable PSC manufacturing process that will produce high yield and quality of target cells. The uniform mixing environment of vertical-wheel bioreactors promotes scalable formation of uniformly spherical cell aggregates, which enables heterogeneous differentiation to be avoided or deterred. Therefore, vertical-wheel bioreactors 102, 106 are a viable tool for large-scale differentiation of PSC aggregates into high quality target cells.

FIG. 26A is a graph 2600 including an X-axis 2602 representing energy dissipation rates (EDRs) and a Y-axis 2604 representing a volume average energy dissipation rate. A first line 2606 represents results associated with using the second bioreactor 106 having a volume of approximately 0.1 L, a second line 2608 represents results associated with using the second bioreactor 106 having a volume of approximately 0.5 L, a third line 2610 represents results using the first bioreactor 102 having a volume of approximately 3 L, and a fourth line 2612 represents results using the first bioreactor 102 having a volume of approximately 15 L. As shown, as the agitation rate increases, the volume average EDR value also increases. The lines 2606, 2608, 2610, 2612 were generated by fitting data points obtained during experiments. Thus, the lines 2606, 2608, 2610 for each agitation rate value and each volume average energy dissipation rate value were determined using a best fit equation to allow each agitation rate for each size containment vessel 104, 108 to have a corresponding volume average EDR value.

Advantageously, using the disclosed examples, an upper average EDR value 2614 and a lower average EDR value 2616 can be determined at which the bioreactors 102, 106 of different sizes can be operated to grow cells having similar sizes and/or diameters. In the example shown, a box 2618 is shown on the graph 2600 that bounds the upper and lower EDR values 2614, 2616 allowing an agitation rate to be selected within the box 2618 and on the corresponding line 2606, 2608, 2610, 2612 for the different volumes that grows cells having threshold characteristics (sizes and/or shapes) and achieve cell growth having threshold characteristics.

FIG. 26B is a graph 2620 including an X-axis 2622 representing the volume of the containment vessel 104, 108 and a Y-axis 2624 representing the agitation rate (RPM). As shown, as the volume increases, the agitation rate decreases. In FIG. 26B, a first line 2680 represents results obtained beginning at an agitation of approximately 40 RPMs, a second line 2682 represents results obtained beginning at an agitation of approximately 50 RPMs, a third line 2684 represents results obtained beginning at an agitation of approximately 60 RPMs, a fourth line 2686 represents results obtained beginning at an agitation of approximately 60 RPMs, and a fifth line 2668 represents results obtained beginning at an agitation of approximately 80 RPMS.

FIG. 26C is a graph 2626 including an X-axis 2628 representing the agitation rate and a Y-axis 2630 representing the average sheer stress. The graph 2626 includes the first line 2606 that represents results associated with using the second bioreactor 106 having a volume of approximately 0.1 L, the second line 2608 represents results associated with using the second bioreactor 106 having a volume of approximately 0.5 L, the third line 2610 represents results using the first bioreactor 102 having a volume of approximately 3 L, and the fourth line 2612 represents results using the first bioreactor 102 having a volume of approximately 15 L. As shown, as the agitation rate increases, the shear stress also increases.

FIG. 26D is a graph 2632 including an X-axis 2634 representing the agitation rate and a Y-axis 2636 representing the volume average velocity. The graph 2626 includes the first line 2606 that represents results associated with using the second bioreactor 106 having a volume of approximately 0.1 L, the second line 2608 represents results associated with using the second bioreactor 106 having a volume of approximately 0.5 L, the third line 2610 represents results using the first bioreactor 102 having a volume of approximately 3 L, and the fourth line 2612 represents results using the first bioreactor 102 having a volume of approximately 15 L. As shown, as the agitation rate, increases the average velocity also increases.

FIG. 26E is a graph 2638 including an X-axis 2640 representing the agitation rate and a Y-axis 2636 representing the volume percent. FIG. 26F shows a more detailed view of a portion of the graph 2638 of FIG. 26E. The graph 2638 includes a first line 2644 that represents results associated with using the bioreactor 102, 106 having a volume of approximately 0.1 L when the wheel 120 is rotated at approximately 60 RPMs, a second line 2646 that represents results associated with using the bioreactor, 102, 106 having a volume of approximately 0.5 L when the wheel 120 is rotated at approximately 30 RPMs, a third line 2648 that represents results using the bioreactor 102 having a volume of approximately 3 L when the wheel 120 is rotated at approximately 20 RPMs, and a fourth line 2650 that represents results using the bioreactor 102, 106 having a volume of approximately 15 L when the wheel 120 is rotated at approximately 13 RPMs. The graph 2638 also includes a fifth line 2655 that represents results using the bioreactor 102, 106 having a volume of approximately 0.5 L when the wheel 120 is rotated at approximately 40 RPMs.

In the example shown, each of the lines 2644, 2646, 2648, 2650 includes actual EDR data points within the volume of the suspension in the bioreactor 102, 106 and, based on the steep negative slope of the lines 2644, 2646, 2648, 2650, a magnitude of a majority of the actual EDR points have an EDR value of less than approximately 0.0015 m²/s³ (see, reference number 2650). For example, for the first line 2644, approximately 97.57% of the EDR values are positioned between approximately 6.1E-04 m²/s³ (see, reference number 2652) and approximately 1.5E-03 m²/s³ (see, reference number 2654). The 97.57% value is determined by adding the 91.51 volume percent value at approximately 6.1E-04 m²/s³ and the 6.06% value at approximately 1.5E-03 m²/s³. For the first line 2644, approximately 97.57% of the EDR values are positioned between approximately 6.1E-04 m²/s³ (see, reference number 2652) and approximately 1.5E-03 m²/s³ (see, reference number 2654). For the second line 2646, approximately 80.78% of the EDR values are positioned between approximately 6.1E-04 m²/s³ and approximately 1.5E-03 m²/s³. For the third line 2648, approximately 91.98% of the EDR values are positioned between approximately 6.1E-04 m²/s³ and approximately 1.5E-03 m²/s³. For the fourth line 2650, approximately 87.18% of the EDR values are positioned between approximately 6.1E-04 m²/s³ and approximately 1.5E-03 m²/s³.

Chart 1 below includes data obtained from experiments using the disclosed implementations. As shown in the chart, approximately 97.57% of the EDR values are positioned between approximately 0.0 m²/s³ and approximately 1.5E-03 m²/s³ when the second bioreactor 106 having a volume of approximately 0.1 L is operated at approximately 60 RPMs, approximately 80.78% of the EDR values are positioned between approximately 0.0 m²/s³ and approximately 1.5E-03 m²/s³ when the second bioreactor 106 having a volume of approximately 0.5 L is operated at approximately 30 RPMs, approximately 62.21% of the EDR values are positioned between approximately 0.0 m²/s³ and approximately 1.5E-03 m²/s³ when the second bioreactor 106 having a volume of approximately 0.5 L is operated at approximately 40 RPMs, approximately 91.98% of the EDR values are positioned between approximately 0.0 m²/s³ and approximately 1.5E-03 m²/s³ when the first bioreactor 102 having a volume of approximately 3.0 L is operated at approximately 20 RPMs, and approximately 87.18% of the EDR values are positioned between approximately 0.0 m²/s³ and approximately 1.5E-03 m²/s³ when the first bioreactor 102 having a volume of approximately 15 L is operated at approximately 13 RPMs.

Additionally, as shown in the chart, approximately 99.80% of the EDR values are positioned between approximately 0.0 m²/s³ and approximately 1.0E-02 m²/s³ when the second bioreactor 106 having a volume of approximately 0.1 L is operated at approximately 60 RPMs, approximately 95.38% of the EDR values are positioned between approximately 0.0 m²/s³ and approximately 1.5E-03 m²/s³ when the second bioreactor 106 having a volume of approximately 0.5 L is operated at approximately 30 RPMs, approximately 87.72% of the EDR values are positioned between approximately 0.0 m²/s³ and approximately 1.5E-03 m²/s³ when the second bioreactor 106 having a volume of approximately 0.5 L is operated at approximately 40 RPMs, approximately 99.14% of the EDR values are positioned between approximately 0.0 m²/s³ and approximately 1.5E-03 m²/s³ when the first bioreactor 102 having a volume of approximately 3.0 L is operated at approximately 20 RPMs, approximately 96.88% of the EDR values are positioned between approximately 0.0 m²/s³ and approximately 1.5E-03 m²/s³ when the first bioreactor 102 having a volume of approximately 15 L is operated at approximately 13 RPMs, and approximately 62.21% of the EDR values are positioned between approximately 0.0 m²/s³ and approximately 1.5E-03 m²/s³ when the second bioreactor 106 having a volume of approximately 0.5 L is operated at approximately 40 RPMs.

CHART 1

| | Total EDR % at Volume, RPM | | | | |
|---|---|---|---|---|---|
| | 0.1 L, | 0.5 L, | 0.5 L, | 3 L, | 15 L, |
| SUM | 60 rpm | 30 rpm | 40 rpm | 20 rpm | 13 rpm |
| 1.50E−03 | 97.57 | 80.78 | 62.21 | 91.98 | 87.18 |
| 1.00E−02 | 99.80 | 95.38 | 87.72 | 99.14 | 96.88 |

In some implementations, at least approximately 60% of the EDR values are less than approximately 1.5E-03 m²/s³ when the second bioreactor 106 is operated, for example, at approximately 40 RPMs while being able to grow cells having similar sizes and/or shapes. For example, in one version approximately 62.21% of the EDR values are less than approximately 1.5E-03 m²/s³ and, more specifically, between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 20% of the EDR values are less than approximately 1.5E-03 m²/s³ when the second bioreactor is being operated, or for example, between approximately 20% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 25% of the EDR values are less than approximately 1.5E-03 m²/s³ when the second bioreactor is being operated, or for example, between approximately 25% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 30% of the EDR values are less than approximately 1.5E-03 m²/s³ when the second bioreactor is being operated, or for example, between approximately 30% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 35% of the EDR values are less than approximately 1.5E-03 m²/s³ when the second bioreactor is being operated, or for example, between approximately 35% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 40% of the EDR values are less than approximately 1.5E-03 m²/s³ when the second bioreactor is being operated, or for example, between approximately 40% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 45% of the EDR values are less than approximately 1.5E-03 m²/s³ when the second bioreactor is being operated, or for example, between approximately 45% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 50% of the EDR values are less than approximately 1.5E-03 m²/s³ when the second bioreactor is being operated, or for example, between approximately 50% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 55% of the EDR values are less than approximately 1.5E-03 m²/s³ when the second bioreactor is being operated, or for example, between approximately 55% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 60% of the EDR values are less than approximately 1.5E-03 m²/s³ when the second bioreactor is being operated, or for example, between approximately 60% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 65% of the EDR values are less than approximately 1.5E-03 m²/s³ when the second bioreactor is being operated, or for example, between approximately 65% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 70% of the EDR values are less than approximately 1.5E-03 m²/s³ when the second bioreactor is being operated, or for example, between approximately 70% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 75% of the EDR values are less than approximately 1.5E-03 m²/s³ when the second bioreactor is being operated, or for example, between approximately 75% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 80% of the EDR values are less than approximately 1.5E-03 m²/s³ when the second bioreactor is being operated, or for example, between approximately 80% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 85% of the EDR values are less than approximately 1.5E-03 m²/s³ when the second bioreactor is being operated, or for example, between approximately 85% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 90% of the EDR values are less than approximately 1.5E-03 m²/s³ when the second bioreactor is being operated, or for example, between approximately 99% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 95% of the EDR values are less than approximately 1.5E-03 m²/s³ when the second bioreactor is being operated, or for example, between approximately 95% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 97% of the EDR values are less than approximately 1.5E-03 m²/s³ when the second bioreactor is being operated, or for example, between approximately 97% and approximately 99% are between approximately 3.0E-04 m²/s³ and approximately 1.5E-03 m²/s³ when the second bioreactor 106 is operated at approximately 40 RPMs while being able to grow cells having similar sizes and/or shapes. In other implementations, at least approximately 99% of the EDR values are less than approximately 1.5E-03 m²/s³ when the second bioreactor is being operated, or for example, at least 99% of the EDR values are between approximately 3.0E-04 m²/s³ and approximately 1.5E-03 m²/s³ when the second bioreactor 106 is operated at approximately 40 RPMs while being able to grow cells having similar sizes and/or shapes.

More generally, a magnitude of at least approximately 60%, at least approximately 65%, at least approximately 70%, at least approximately 75%, at least approximately 80%, at least approximately 85%, at least approximately 90%, at least approximately 95%, at least approximately 97%, or at least approximately 99% of the plurality of actual EDR data points is less than approximately 0.0015 m²/s³. In other implementations, a percentage of actual EDR data points being (a) less than approximately 0.0015 m²/s³, (b) less than approximately 0.002 m²/s³, (c) less than approximately 0.0025 m²/s³, or (d) less than approximately 0.003 m²/s³ in the second bioreactor during operation of the second bioreactor is in a range of approximately 60% and approximately 99%, approximately 60% and approximately 97%, approximately 60% and approximately 95%, approximately 60% and approximately 90%, approximately 60% and approximately 85%, approximately 60% and approximately 80%, approximately 60% and approximately 75%, approximately 60% and approximately 70%, approximately 60% and approximately 65%, approximately 65% and approximately 99%, approximately 65% and approximately 97%, approximately 65% and approximately 95%, approximately 65% and approximately 90%, approximately 65% and approximately 85%, approximately 65% and approximately 80%, approximately 65% and approximately 75%, approximately 65% and approximately 70%, approximately 70% and approximately 99%, approximately 70% and approximately 97%, approximately 70% and approximately 95%, approximately 70% and approximately 90%, approximately 70% and approximately 85%, approximately 70% and approximately 80%, approximately 70% and approximately 75%, approximately 75% and approximately 99%, approximately 75% and approximately 97%, approximately 75% and approximately 95%, approximately 75% and approximately 90%, approximately 75% and approximately 85%, approximately 75% and approximately 80%, approximately 80% and approximately 99%, approximately 80% and approximately 97%, approximately 80% and approximately 95%, approximately 80% and approximately 90%, approximately 80% and approximately 85%, approximately 85% and approximately 99%, approximately 85% and approximately 97%, approximately 85% and approximately 95%, approximately 85% and approximately 90%, approximately 90% and approximately 99%, approximately 90% and approximately 97%, approximately 90% and approximately 95%, approximately 95% and approximately 99%, or approximately 95% and approximately 97%.

In other implementations, a percentage of actual EDR data points being between (a) approximately 3.0E-04 m²/s³ and approximately 1.5E-03 m²/s³, (b) approximately 3.0E-04 m²/s³ and approximately 0.002 m²/s³, (c) approximately 3.0E-04 m²/s³ and 0.0025 m²/s³, or (d) approximately 3.0E-04 m²/s³ and approximately 0.003 m²/s³ in the second bioreactor during operation of the second bioreactor is in a range of approximately 60% and approximately 99%, approximately 60% and approximately 97%, approximately 60% and approximately 95%, approximately 60% and approximately 90%, approximately 60% and approximately 85%, approximately 60% and approximately 80%, approximately 60% and approximately 75%, approximately 60% and approximately 70%, approximately 60% and approximately 65%, approximately 65% and approximately 99%, approximately 65% and approximately 97%, approximately 65% and approximately 95%, approximately 65% and approximately 90%, approximately 65% and approximately 85%, approximately 65% and approximately 80%, approximately 65% and approximately 75%, approximately 65% and approximately 70%, approximately 70% and approximately 99%, approximately 70% and approximately 97%, approximately 70% and approximately 95%, approximately 70% and approximately 90%, approximately 70% and approximately 85%, approximately 70% and approximately 80%, approximately 70% and approximately 75%, approximately 75% and approximately 99%, approximately 75% and approximately 97%, approximately 75% and approximately 95%, approximately 75% and approximately 90%, approximately 75% and approximately 85%, approximately 75% and approximately 80%, approximately 80% and approximately 99%, approximately 80% and approximately 97%, approximately 80% and approximately 95%, approximately 80% and approximately 90%, approximately 80% and approximately 85%, approximately 85% and approximately 99%, approximately 85% and approximately 97%, approximately 85% and approximately 95%, approximately 85% and approximately 90%, approximately 90% and approximately 99%, approximately 90% and approximately 97%, approximately 90% and approximately 95%, approximately 95% and approximately 99%, or approximately 95% and approximately 97%.

In other implementations, a magnitude of at least approximately 60%, at least approximately 65%, at least approximately 70%, at least approximately 75%, at least approximately 80%, at least approximately 85%, at least approximately 90%, at least approximately 95%, at least approximately 97%, or at least approximately 99% of the plurality of actual EDR data points has an energy dissipation rate value of less than approximately 0.002 m²/s³, less than approximately 0.0025 m²/s³, or less than approximately 0.003 m²/s³.

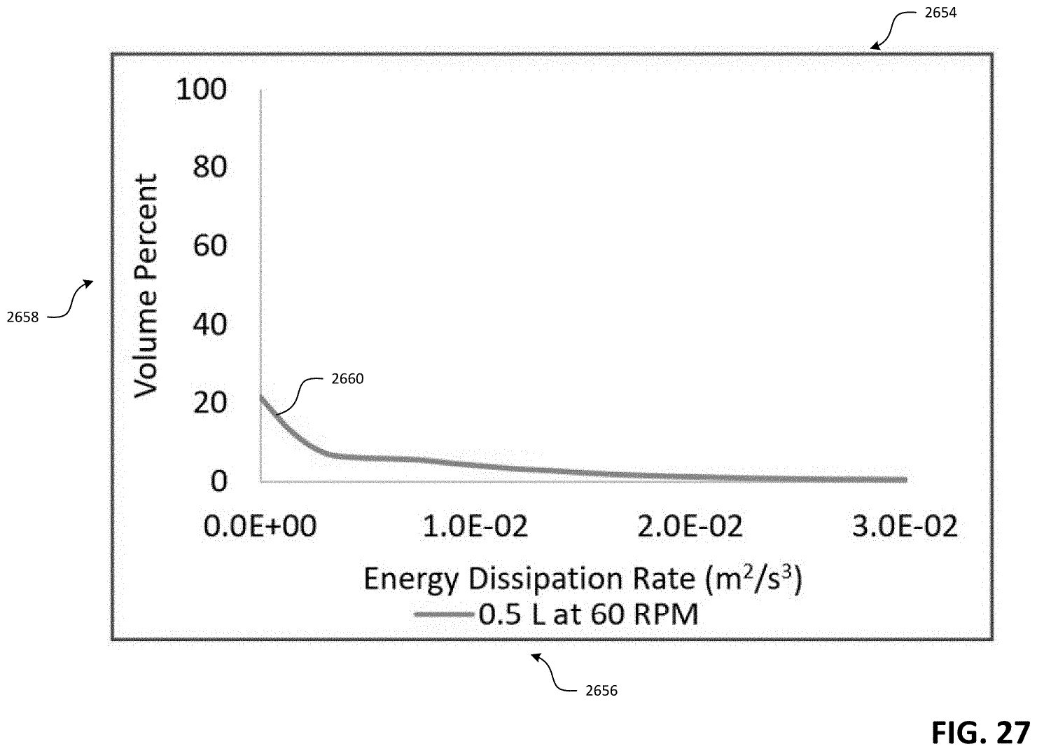

FIG. 27 is a graph 2654 including an X-axis 2656 representing the agitation rate and a Y-axis 2658 representing the volume percent. The graph 2638 includes a first line 2660 that represents results associated with using a bioreactor having a volume of approximately 0.5 L with a wheel that is rotated at approximately 60 RPMs. The bioreactor may be a horizontal bioreactor or a vertical bioreactor. As shown, a slope of the first line 2660 is relatively gradual as compared to the slopes of the lines 2644, 2646, 2648, 2650 of FIG. 26E and is associated with an agitation rate (see, FIG. 26A) that is outside of the box 2614. As such, cells grown in such a bioreactor with the agitation rate of 60 RPMs may not have similar shapes and/or sizes.

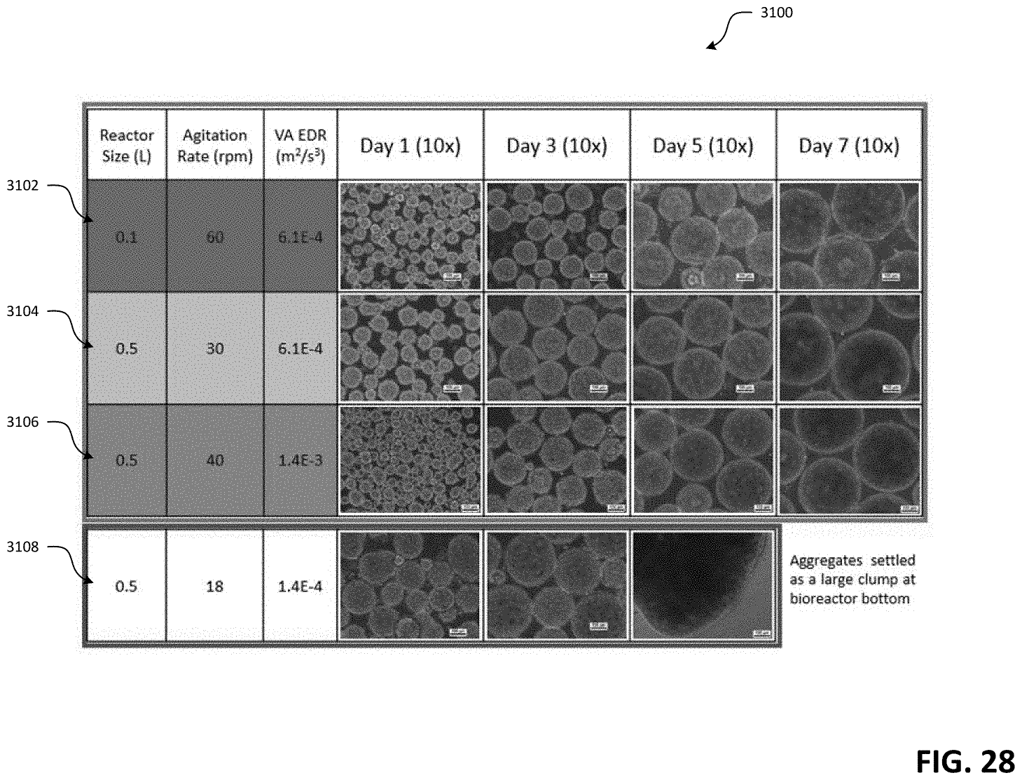

FIG. 28 shows the biological results 3100 obtained through the combination of having target VA EDR inside a threshold range, as well as having majority or at least some of EDR values below the upper threshold value of approximately 1.5E-03 m²/s³. In the results, a first row 3102 and a second row 3102 indicate agitation rates to achieve a desired target VA EDR of approximately 6.1E-04 m²/s³ at two different volumes of using the second bioreactor 106. Visually, distribution of aggregate sizes and shapes are similar, even from Day 1. A third row 3106 represents different target VA EDR of approximately 1.4E-03 m²/s³ and the correspondingly higher agitation rate in 0.5 L bioreactor. Initially on Day 1, aggregates are comparatively smaller in size, but after Day 3 the aggregates look similar to those formed with lower target VA EDR (that is still within the threshold range). A fourth row 3108 is example of target VA EDR of approximately 1.4E-04 m²/s³ that is outside (below) the threshold range. As a result of being outside of the threshold range, aggregates have more variation in size and/or shape, and after Day 7 the aggregates have clumped together so much they cannot or have a lesser tendency to suspend in liquid. Also, for each combination of bioreactor volume and agitation rate, there is corresponding % of all EDR values that fall within the threshold range (approximately 98%, 81%, 62% for rows 1, 2, 3, respectively). Homogenous or substantially homogenous distribution of aggregates, of similar size and shape, was achieved using all three of these hydrodynamic conditions.

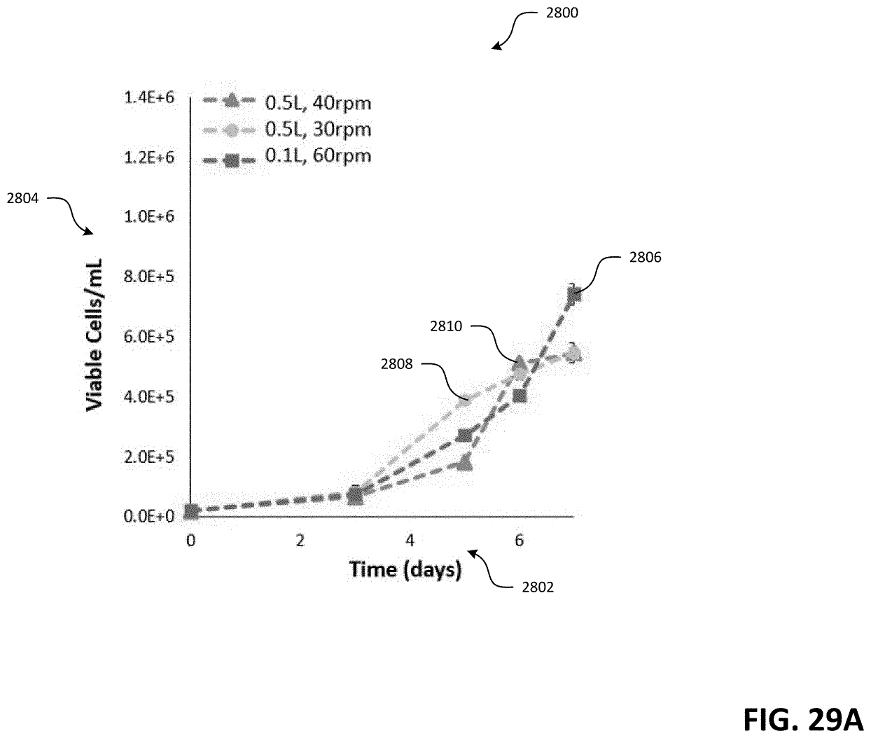

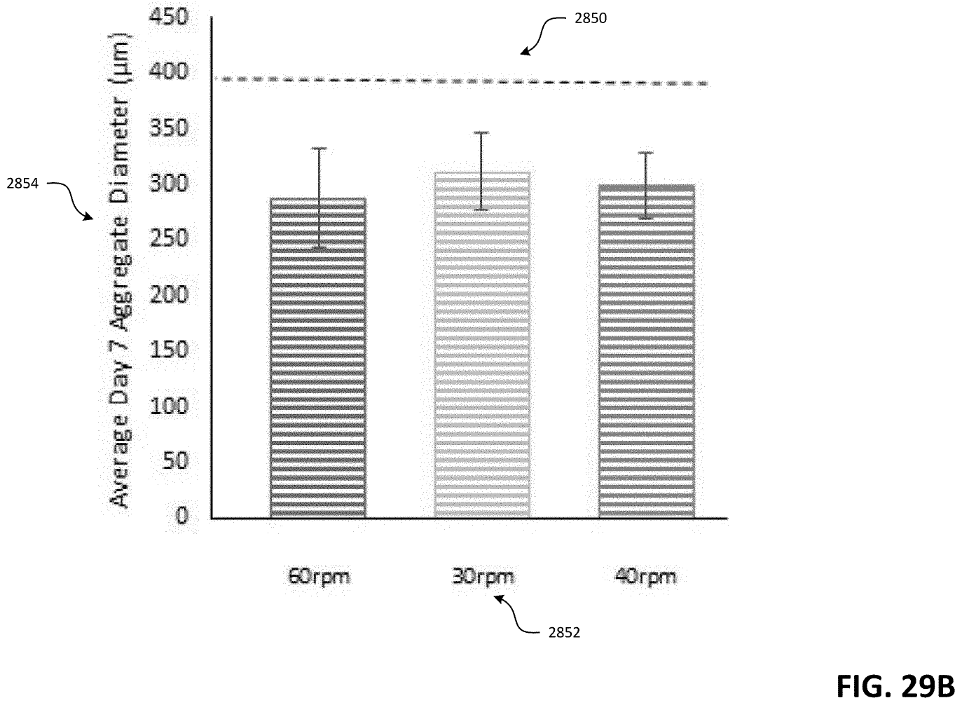

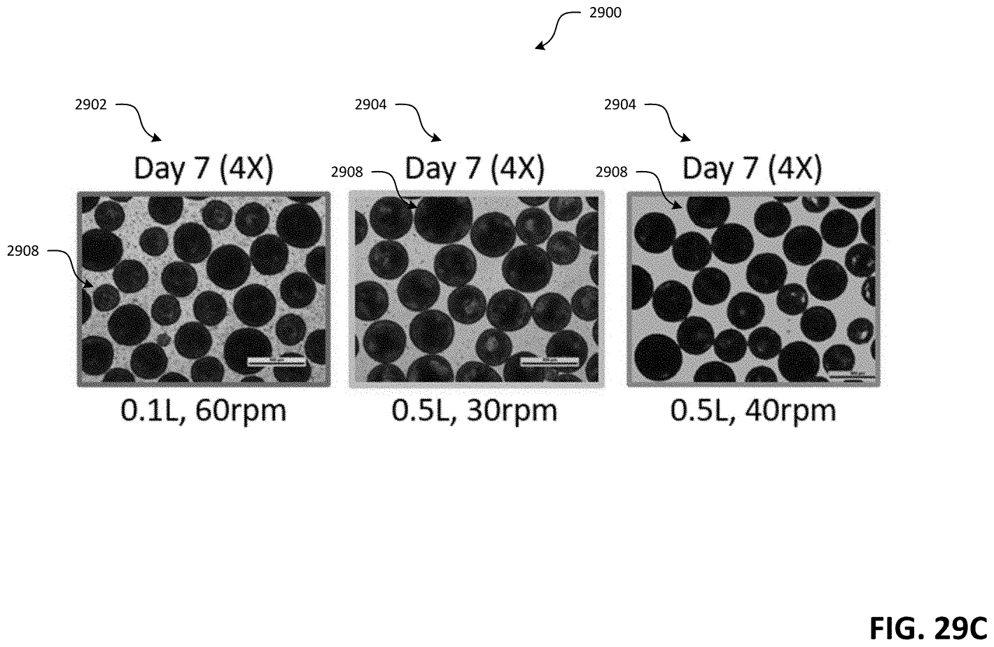

FIG. 29A is a graph 2800 including an X-axis 2802 representing time in days and a Y-axis 2804 representing viable cells in mL. The graph 2800 includes a first line 2806 that represents results associated with using the bioreactor 102, 106 having a volume of approximately 0.1 L when the wheel 120 is rotated at approximately 60 RPMs, a second line 2808 that represents results associated with using the bioreactor 102, 106 having a volume of approximately 0.5 L when the wheel 120 is rotated at approximately 30 RPMs, and a third line 2810 that represents results associated with using the bioreactor 102, 106 having a volume of approximately 0.5 L when the wheel 120 is rotated at approximately 40 RPMs. As shown, as the number of days increase, the number of viable cells also increase.

FIG. 29B is a graph 2850 representing results from the experiments performed in association with FIG. 29A and includes an X-axis 2852 representing the agitation rates at which the wheel 120 of the second bioreactor 106 was operated and a y-axis 2854 representing the average day 7 aggregate diameter. Advantageously and as shown, the diameter of the cells grown using the second bioreactor 106 at the different agitation rates are relatively similar and between approximately 270 microns and approximately 320 microns.

FIG. 29C are image results 2900 representing results from the experiments performed in association with FIGS. 28A and 28B. As shown, images 2902, 2904, and 2906 each have cells 2908 that have substantially similar shapes and/or sizes after the cells have been grown for 7 days. The results 2900 indicate that the aggregates are statistically similar in average diameter, with overlapping single standard deviation error bars.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

1. A method of scaling production of therapeutic cells grown on microcarriers or as cell aggregates in a suspension-based bioreactor, the method comprising:
determining a target average energy dissipation rate (EDR) of turbulent eddies within a suspension including cells disposed in a small scale bioreactor;
determining a small scale agitation rate to achieve the target average EDR in the small scale bioreactor;
determining a large scale agitation rate to achieve the target average EDR in a large scale bioreactor, the large scale agitation rate being directly dependent on the small scale agitation rate;
depositing a suspension comprising a plurality of cells suspended in a volume of culture fluid into the large scale bioreactor;
setting an agitation rate of a mixer disposed in the large scale bioreactor to the large scale agitation rate; and
actuating the mixer in the large scale bioreactor at the large scale agitation rate to mix the suspension with an average EDR approximately equal to the target average EDR.

2. The method of claim 1, wherein the average EDR comprises an average of a plurality of actual EDR data points within the volume of the suspension in the large scale bioreactor, wherein a magnitude of at least approximately 60%, at least approximately 70%, at least approximately 75%, at least approximately 80%, at least approximately 85%, at least approximately 90%, at least approximately 95%, or at least approximately 97% of the plurality of actual EDR data points is less than approximately 0.0015 m²/s³.

3. The method of of claim 1, wherein at least one of the small scale and large scale agitation rates is in a range between approximately 0 rpm and approximately 120 rpm.

4. The method of of claim 1, wherein at least one of the small scale and large scale agitation rates are in a range between approximately 12 rpm and approximately 77 rpm.

5. The method of claim 1, wherein the target average EDR is in a range between approximately 0 m²/s³ and approximately 0.006 m²/s³.

6. The method of claim 1, wherein the target average EDR is in a range between approximately 0.0003 m²/s³ and approximately 0.0015 m²/s³.

7. The method of claim 1, wherein actuating the mixer in the large scale bioreactor comprises actuating a vertical wheel mixer having a horizontal axis of rotation.

8. The method of claim 1, wherein actuating the mixer in the large scale bioreactor comprises actuating a mixer having a vertical axis of rotation.

9. The method of claim 1, wherein depositing a suspension including cells into the large scale bioreactor comprises depositing pluripotent stem cells (PSCs) into the large scale bioreactor.

10. The method of claim 1, further comprising depositing microcarriers into the large scale bioreactor.

11. The method of claim 1, wherein the large scale bioreactor has a volume larger than a volume of the small scale bioreactor.

12. A method of operating a large scale suspension-based bioreactor for the production of cells grown on microcarriers or as cell aggregates, the method comprising:
selecting a large scale bioreactor for production of cells grown on microcarriers or as cell aggregates, the large scale bioreactor having a large scale mixer in a large scale vessel,
determining a large scale agitation rate for the large scale mixer, the large scale agitation rate being determined based on a small scale agitation rate of a small scale mixer in a small scale vessel of a small scale bioreactor that achieves a target average energy dissipation rate (EDR) of turbulent eddies in a suspension in the small scale bioreactor,
depositing a suspension comprising cells suspended in a volume of culture fluid into the large scale bioreactor,
setting the agitation rate of the large scale mixer to the large scale agitation rate,
actuating the large scale mixer at the large scale agitation rate to mix the cells in the suspension at an average EDR approximately equal to the target average EDR.

13. The method of claim 12, wherein the average EDR comprises an average of a plurality of actual EDR data points within the volume of the suspension in the large scale bioreactor, wherein a magnitude of at least approximately 60%, at least approximately 70%, at least approximately 75%, at least approximately 80%, at least approximately 85%, at least approximately 90%, at least approximately 95%, or at least approximately 97% of the plurality of actual EDR data points is less than approximately 0.0015 m²/s³.

14. The method of claim 12, wherein at least one of the small scale and large scale agitation rates is in a range between approximately 0 rpm and approximately 120 rpm.

15. The method of claim 12, wherein at least one of the small scale and large scale agitation rates is in a range between approximately 12 rpm and approximately 77 rpm.

16. The method of claim 12, wherein the target average EDR is in a range between approximately 0 m²/s³ and approximately 0.006 m²/s³.

17. The method of claim 12, wherein the target average EDR is in a range between approximately 0.003 m²/s³ and approximately 0.0015 m²/s³.

18. The method of claim 12, wherein actuating the large scale mixer comprises a vertical wheel mixer having a horizontal axis of rotation.

19. The method of claim 12, wherein actuating the large scale mixer comprises actuating a mixer having a vertical axis of rotation.

20. The method of claim 12, wherein depositing a suspension including cells into the large scale bioreactor comprises depositing pluripotent stem cells (PSCs) into the large scale bioreactor.

21. The method of claim 12, further comprising depositing microcarriers into the large scale bioreactor.

22. The method of claim 12, wherein selecting a large scale bioreactor comprises selecting a bioreactor with a volume larger than a volume of the small scale bioreactor.

23. A large scale suspension-based system for the production of cells grown on microcarriers or as cell aggregates, the system comprising:
a bioreactor comprising a vessel and a mixer disposed in the vessel, the mixer operably coupled to a drive mechanism and being operated at an agitation rate;
a suspension comprising cells suspended in a volume of culture fluid disposed in the vessel and being mixed by the mixer, the suspension including a plurality of turbulent eddies generated by the mixer, the plurality of turbulent eddies each having an energy dissipation rate (EDR), wherein a magnitude of the EDR of at least approximately 60%, at least approximately 70%, at least approximately 75%, at least approximately 80%, at least approximately 85%, at least approximately 90%, at least approximately 95%, or at least approximately 97% of the turbulent eddies is less than approximately 0.0015 m²/s³.

24. The system of claim 23, wherein the target average EDR is in a range between approximately 0 m²/s³ and approximately 0.006 m²/s³.

25. The system of claim 23, wherein the target average EDR is in a range between approximately 0.0003 m²/s³ and approximately 0.0015 m²/s³.

26. The system of claim 23, wherein the vessel has a volume at least one of between approximately 0.1 L and approximately 500 L or between approximately 0.1 L and approximately 2000 L.

27. The system of claim 23, wherein the mixer comprises a vertical wheel mixer having a horizontal axis of rotation.

28. The system of claim 23, wherein the vessel comprises a curved bottom wall.

29. The system of claim 23, wherein the mixer comprises a vertical axis of rotation.

30. The system of claim 23, wherein the cells comprise pluripotent stem cells (PSCs).

31. The system of claim 23, further comprising microcarriers in the suspension.

32. Method of production of therapeutic cells grown on microcarriers or as cell aggregates in a suspension-based bioreactor, the method comprising:
depositing a suspension comprising cells suspended in a volume of culture fluid into a bioreactor,
setting an agitation rate of a mixer disposed in the bioreactor,
actuating the mixer at the set agitation rate to mix the suspension in the bioreactor, the suspension including a plurality of turbulent eddies generated by the mixer, wherein a magnitude of an energy dissipation rate (EDR) of at least approximately 60%, at least approximately 70%, at least approximately 75%, at least approximately 80%, at least approximately 85%, at least approximately 90%, at least approximately 95%, or at least approximately 97% of the turbulent eddies is less than approximately 0.0015 m²/s³.

33. The method of claim 32, wherein the target average EDR is in a range between approximately 0 m²/s³ and approximately 0.006 m²/s³.

34. The method of claim 32, wherein the target average EDR is in a range between approximately 0.0003 m²/s³ and approximately 0.0015 m²/s³.

35. The method of claim 32, further comprising selecting the bioreactor from a plurality of available bioreactors, each comprising a volume of at least one of between approximately 0.1 L and approximately 500 L or between approximately 0.1 L and approximately 2000 L.

36. The method of claim 32, wherein the mixer comprises a vertical wheel mixer having a horizontal axis of rotation.

37. The method of claim 32, wherein the vessel comprises a curved bottom wall.

38. The method of claim 32, wherein the mixer comprises a vertical axis of rotation.

39. The method of claim 32, wherein depositing a suspension comprising cells suspended in a volume of a culture fluid into the bioreactor comprises depositing pluripotent stem cells (PSCs) into the bioreactor.

40. The method of claim 32, further comprising depositing microcarriers into the bioreactor.

41-54. (canceled)
